(19) United States
(12) Patent Application Publication
(10) Pub. No.: US 2017/0035962 A1
(43) Pub. Date: Feb. 9, 2017

(54) INFUSION SYSTEM AND METHODS

(71) Applicant: Tandem Diabetes Care, Inc., San Diego, CA (US)

(72) Inventors: Aymeric A. Lecanu-Fayet, Carlsbad, CA (US); Vance Swanson, San Diego, CA (US); Sean Saint, San Diego, CA (US)

(21) Appl. No.: 15/294,845

(22) Filed: Oct. 17, 2016

Related U.S. Application Data
(63) Continuation of application No. 13/827,383, filed on Mar. 14, 2013.

(51) Int. Cl. A61M 5/142; A61M 5/162

(52) U.S. Cl. CPC A61M 5/14244; A61M 5/162

(57) ABSTRACT

An infusion cartridge for use with an ambulatory infusion system can be provided with a preattached infusion set to eliminate potential leakage, potential snagging and other inconveniences from tubing connectors. The infusion cartridge can include a rigid housing, a collapsible fluid reservoir within the housing and a dispense mechanism for drawing fluid from the reservoir and sending it out an outlet port. A length of tubing can be fixedly attached to the outlet port in fluid communication with the outlet port. The length of tubing can extend as a unitary construct from the outlet port to a connector that connects to a cannula for delivering fluid from the reservoir to a user.

### RELATED APPLICATION

This application is a continuation of application Ser. No. 13/827,383 filed Mar. 14, 2013, which is hereby fully incorporated herein by reference.

### FIELD OF THE INVENTION

The present invention is directed to infusion devices or systems and methods of using these devices or systems for dispensing fluids in a controllable and reliable manner. In some cases, embodiments may include portable infusion pumps and methods of using such pumps for infusing a fluid material or multiple fluid materials, such as liquid medicaments (including drugs or therapeutic fluids), to a patient or any other suitable destination.

### BACKGROUND

There are many applications in academic, industrial, and medical fields, as well as others, that benefit from devices and methods that are capable of accurately and controllably delivering fluids, including liquids and gases that have a beneficial effect when administered in known and controlled quantities. Such devices and methods are particularly useful in the medical field where treatments for many patients include the administration of a known amount of a substance at predetermined intervals. In some instances, it may be desirable to safely, reliably, and comfortably administer required doses of more than one medication or other fluid to a patient from a single device.

Insulin-injecting pumps have been developed for the administration of insulin for those suffering from both type I and type II diabetes. Recently, continuous subcutaneous insulin injection and/or infusion therapy with portable infusion devices has been adapted for the treatment of diabetes. Such therapy may include the regular and/or continuous injection or infusion of insulin into the skin of a person suffering from diabetes and offer an alternative to multiple daily injections of insulin by an insulin syringe or an insulin pen. Such pumps can be ambulatory/portable infusion pumps that are worn by the user and may use replaceable cartridges. Examples of such pumps and various features that can be associated with such pumps include those disclosed in U.S. patent application Ser. No. 13/557,163, U.S. patent application Ser. No. 12/714,299, U.S. patent application Ser. No. 12/538,018, U.S. Provisional Patent Application No. 61/655,883, U.S. Provisional Patent Application No. 61/656,967 and U.S. Pat. No. 8,287,495, each of which is incorporated herein by reference. Diabetic patients can also benefit from the administration of other medicaments, such as for, example, glucagon, pramlintide and liraglutide.

What have been needed are devices and methods capable of reliably delivering two or more therapeutic substances from a single device in an efficient manner. Such devices and methods may be useful in some cases in order to facilitate patient compliance, accurate treatment of medical conditions requiring multiple medicaments as well as other applications or indications.

### SUMMARY

An infusion cartridge for use with an ambulatory infusion system can be provided with a preattached infusion set to eliminate potential leakage, potential snagging and other inconveniences from tubing connectors. The infusion cartridge can include a rigid housing, a collapsible fluid reservoir within the housing and a dispense mechanism for drawing fluid from the reservoir and sending it out an outlet port. A length of tubing can be fixedly attached to the outlet port in fluid communication with the outlet port. The length of tubing can extend as a unitary construct from the outlet port to a connector that connects to a cannula for delivering fluid from the reservoir to a user.

In some embodiments, an infusion apparatus include an infusion cartridge comprising a rigid housing; a collapsible fluid reservoir formed from flexible material and disposed within the rigid housing; a dispense mechanism fluidly connected to the fluid reservoir; and an outlet port fluidly connected to the dispense mechanism. The infusion apparatus may also include a length of tubing fixedly connected to the outlet port of the infusion cartridge in fluid communication with the outlet port. A connector can be operably connected to the length of tubing at a terminus of the tubing with the tubing forming a unitary construct from the outlet port of the infusion cartridge to the connector, wherein the connector allows a fluid drug to flow from the infusion cartridge, through the length of tubing, and into a cannula and/or needle.

In some embodiments of an infusion kit, the kit includes a cannula having a first end configured to pierce a patient's skin and a second end. The infusion kit also includes an infusion cartridge including a rigid housing, a collapsible fluid reservoir formed from a flexible material and disposed within the rigid housing, a dispense mechanism fluidly connected to the fluid reservoir, and an outlet port fluidly connected to the dispense mechanism. A length of tubing is fixedly attached to the outlet port of the infusion cartridge. The infusion kit may also include a connector operably connected to the length of tubing at a terminus of the tubing such that the tubing extends as a unitary structure between the outlet port and the connector. The connector can be configured to allow a fluid drug to flow from the infusion cartridge through the length of tubing and into the cannula via the second end of the cannula.

Certain embodiments are described further in the following description, examples, claims, and drawings. These features of embodiments will become more apparent from the following detailed description when taken in conjunction with the accompanying exemplary drawings.

### BRIEF DESCRIPTION OF THE DRAWINGS

FIG. 1 depicts an embodiment of an infusion system according to an embodiment of the present invention in operative communication with a patient.

FIG. 2 depicts an enlarged view of an infusion set with a distal end of an infusion line of the infusion system of FIG. 1 disposed subcutaneously in the patient.

FIG. 2A depicts an enlarged view an infusion set with a distal end of an infusion line of the infusion system of FIG. 1 within and in fluid communication with an interior volume of a vessel of the patient.

FIG. 3 depicts an embodiment of a dual-lumen cannula of the infusion set of FIG. 2.

FIG. 4 depicts another embodiment of a dual-lumen cannula of the infusion set of FIG. 2.

FIG. 5A depicts a partial top view of an embodiment of a dual-lumen cannula of the infusion set of FIG. 2.

FIG. 5B depicts a side view of the dual-lumen cannula of the infusion set of FIG. 5A.

FIG. 5C depicts a cross-sectional view of the dual-lumen cannula of the infusion set of FIG. 5A.

FIG. 6A depicts a cross-sectional view of an embodiment of a dual-lumen cannula of the infusion set of FIG. 2.

FIG. 6B depicts another cross-sectional view of the dual-lumen cannula of FIG. 6A taken along the line of 6B-6B of FIG. 6A.

FIG. 6C depicts a cross-sectional view of an embodiment of a dual-lumen cannula of the infusion set of FIG. 2.

FIG. 6D depicts a cross-sectional view of the dual-lumen cannula of FIG. 6C taken along the line of 6D-6D of FIG. 6C.

FIG. 6E depicts an infusion cartridge and an infusion set with a short length of tubing and a male Luer lock fitting according to the prior art.

FIG. 6F depicts an infusion cartridge with a pre-attached tubing according to an embodiment of the present invention.

FIG. 6G depicts a tubing with coaxial lumina according to an embodiment of the present invention.

FIG. 6H depicts two sections of tubing joined together along their sides before a connector according to an embodiment of the present invention.

FIG. 6J depicts a tubing that is split into two sections of tubing forming a “Y” shape according to an embodiment of the present invention.

FIG. 6K depicts a tubing with coaxial lumina that is split into two sections of tubing forming a “Y” shape according to an embodiment of the present invention.

FIG. 6L depicts two sections of tubing joined together along their sides and bifurcated into two of sections of tubing forming a “Y” shape according to an embodiment of the present invention.

FIG. 7 depicts an embodiment of an infusion pump assembly according to an embodiment of the present invention.

FIG. 8 depicts an embodiment of the infusion pump of FIG. 7 having an infusion cartridge coupled thereto.

FIG. 9 depicts a block diagram representing the infusion pump of FIG. 8.

FIG. 10 depicts a perspective view of the infusion pump of FIG. 8 having a graphic user interface with touch screen capability.

FIG. 11 is an exploded view of the infusion cartridge and infusion pump of FIG. 8 with the cartridge removed from the infusion pump.

FIG. 12 is a schematic view partially cut away depicting some internal components of the infusion pump embodiment of FIG. 8 including a disposable cartridge with a delivery mechanism.

FIG. 13 is an elevation view in partial section of a delivery mechanism embodiment of FIG. 12 coupled to a drive mechanism embodiment.

FIG. 14A depicts an embodiment of a delivery mechanism having two variable volume elements configured to fill from two separate cartridge reservoirs according to an embodiment of the present invention.

FIGS. 14B-14D depict a delivery sequence of the variable volume elements of the delivery mechanism embodiment of FIG. 14A, with the delivery sequence initiating at FIG. 14A.

FIGS. 15A-15C depict a venting sequence of the delivery mechanism embodiment of FIG. 14A with full variable volume elements.

FIGS. 16A-16B depict a delivery sequence of one of the variable volume elements of the delivery mechanism embodiment of FIG. 14A.

FIGS. 16C-16D depict a delivery sequence of one of the variable volume elements of the delivery mechanism embodiment of FIG. 14A.

FIGS. 16E-16F depict a return sequence embodiment for the delivery mechanism embodiment of FIG. 14A.

### DETAILED DESCRIPTION

Some infusion device, system, and method embodiments discussed herein may be used to account for a wide range of variables in determining an amount of medicament, e.g., insulin, to be infused into a patient over a given period of time. Further, some embodiments discussed herein may allow for fine regulation of the amount of medicament delivered, as well as, the time during which the medicament is delivered. Some embodiments may include advances in both the internal components and the control circuitry, as well as, improvements in a user interface. The advances may allow for a more accurate regulation of blood glucose levels than currently attainable by the devices, systems, and methods that are available at this time. Although embodiments described herein may be discussed in the context of the controlled delivery of medicaments such as insulin, other indications and applications are also contemplated. Some liquid medicaments suitable for delivery to a patient by device and method embodiments discussed herein may include insulin, antibiotics, glucose, saline, anesthetics, steroids, opiods, blood thinners, anti-infective agents, glucagon, pramlintide and liraglutide. Device and method embodiments discussed herein may also be used for pain medication, chemotherapy, iron chelation, immunoglobulin treatment, dextrose or saline IV delivery, or any other suitable indication or application. Non-medical applications are also contemplated.

Some embodiments discussed herein may include an interchangeable pump assembly that provides a user with the flexibility and convenience to alternate between pump devices having various features and advantages at any given moment during a single treatment protocol. In some cases a single insulin cartridge may be switched between pump devices such as a smaller more discreet pumping device having fewer features and a larger pumping device having more features during a single treatment without compromising the sterility and thus wasting the cartridge.

As discussed above, there are many applications in academic, industrial, and medical fields, as well as others that may benefit from devices and methods that are capable of accurately and controllably delivering fluids, including liquids and gases that have a beneficial effect when administered in known and controlled quantities. It may be particularly desirable to safely, reliably, and comfortably administer required doses of more than one medication or other fluid to a patient from a single device. This may arise in cases where a treatment regimen involving multiple therapeutic substances is coordinated by a single processor or controller which may also be included in a single device, though multiple conduits to the patient's body may be required.

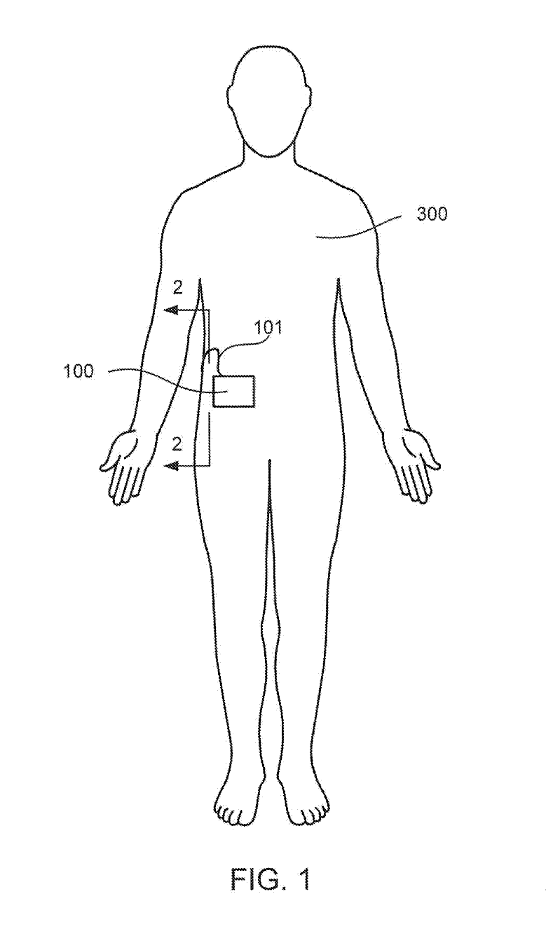

FIG. 1 depicts an infusion system embodiment where an infusion pump system 100 is operatively coupled to an infusion set that is attached to a patient 300. The infusion set may include one or more sections of fluid tubing 101. Distal ends of inner lumens of fluid tubing 101 may be connected to and in fluid communication with respective outlet ports of the infusion pump system 100. Proximal ends of the fluid tubing may be connected to and in fluid communication with respective inlet ports of the infusion set. Exemplary infusion sets include the Cleo® 90 infusion set sold by Smiths Medical ASD, Inc. and the Quick-Set®, Silhouette®, Mio® and SureT® infusion sets sold by Medtronic Minimed, Inc. Examples of infusion pump systems that can be used with embodiments disclosed herein include U.S. patent application Ser. No. 13/557,163, U.S. patent application Ser. No. 12/714,299, U.S. patent application Ser. No. 12/538,018, U.S. Provisional Patent Application No. 61/655,883, U.S. Provisional Patent Application No. 61/656,967 and U.S. Pat. No. 8,287,495, each of which is incorporated herein by reference.

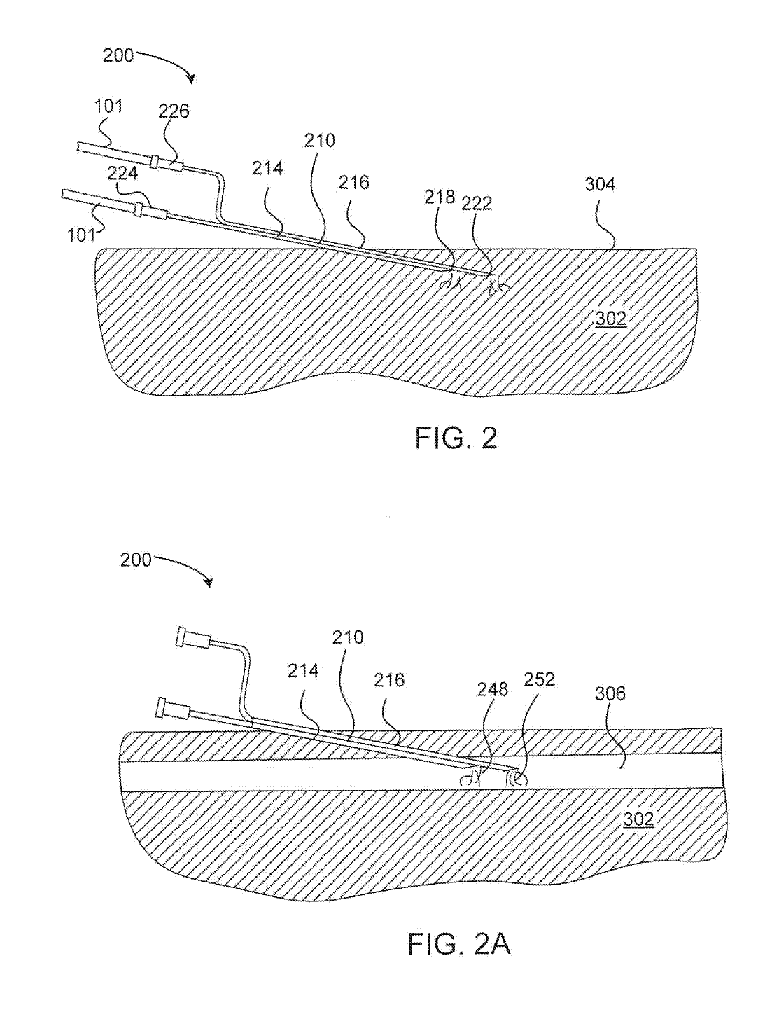

FIG. 2 depicts an infusion set 200 with a dual-lumen cannula 210 at distal ends of fluid tubing 101 of an infusion set 200. The dual-lumen cannula 210 may be inserted in a subcutaneous space under the patient's skin 304. The infusion set 200 is in fluid communication with a dispense port of the infusion pump system 100, and fluids such as insulin and other suitable medicaments, are shown being dispensed from outlet ports 218 and 222 into the patient's body 302. The distal ends of the fluid tubing may be held in place by a piece of an adhesive pad (not shown) secured to the patient's skin 304. The dual-lumen cannula 210 may also be disposed within an inner lumen of a patient's fluid vessel 306, such as, e.g., shown in FIG. 2A. Therapeutic fluids 248 and 252 may be dispensed from outlet ports 218 and 222 respectively to the patient's fluid vessel 306, such as, e.g., a vein, artery, duct, or other vessel. One or both inner lumens of the dual-lumen cannula 210 may be in fluid communication with the inner lumen of the patient's fluid vessel 306 and may also be used in some cases to withdraw fluids from the patient's body 302 back towards the infusion pump system 100 for analysis or the like. The proximal ends of the infusion set 200 may be in fluid communication with a dispense port, such as, e.g., an outlet port of a delivery mechanism of any of the infusion pump system embodiments discussed herein. Any of the infusion pump embodiments discussed herein may be coupled to a patient's body 302 as shown in FIGS. 1-2A.

The infusion set 200 may be configured to be attached to a patient's skin 304 and to be coupled to the infusion pump system 100. In some cases, the dual-lumen cannula 210 may include a first side, with a first cannula 214 configured to provide the first therapeutic fluid 248. The second side may include a second cannula 216 configured to provide the second therapeutic fluid 252.

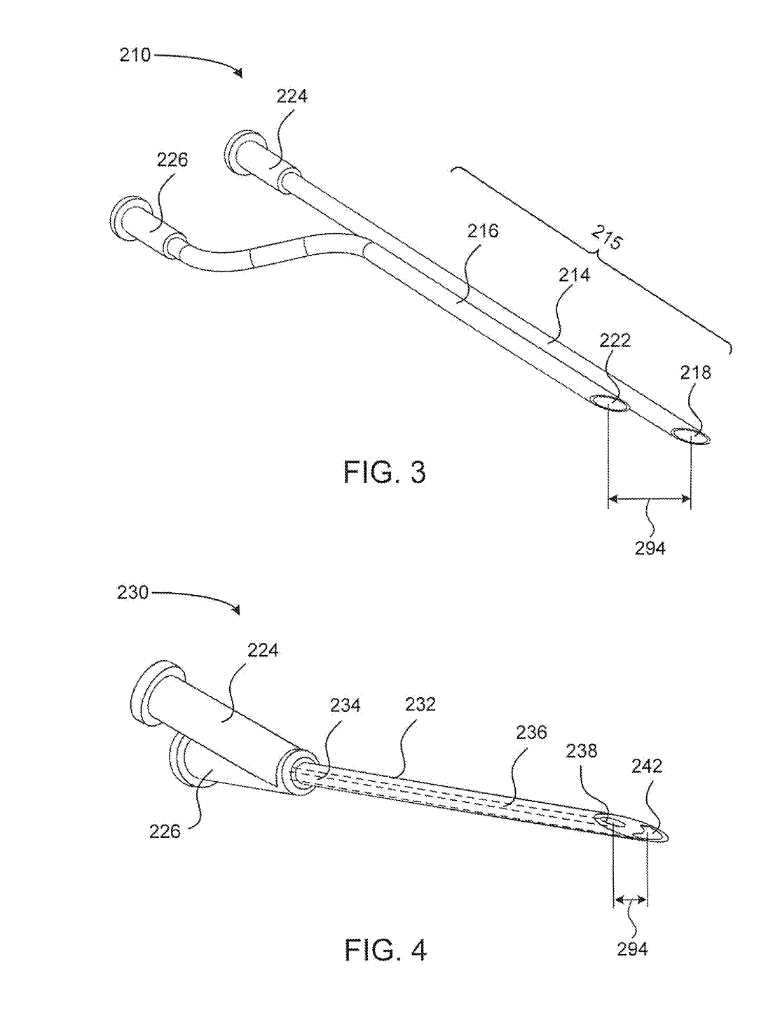

FIG. 3 shows a perspective view of the dual-lumen cannula 210 of the infusion set 200 of FIG. 2 according to an embodiment of the present invention. The dual-lumen cannula 210 may include the first and the second cannulas 214 and 216. The first and the second cannulas 214 and 216 of the dual-lumen cannula 210 may be joined together as a single cannula assembly with at least two lumens. The first lumen and the second lumen may have about the same diameters in some cases. The first cannula 214 and the second cannula 216 may have separate first and second proximal ends respectively. A first cannula inlet port 224 and a second cannula inlet port 226 may be formed at the first and the second proximal ends of the cannula 210, respectively. A length of the first cannula 214 may be greater than a length of the second cannula 216 such that openings at the distal ends, i.e. output ports, of the first cannula 214 and the second cannula 216 may be axially offset from each other. An offset 294 between the output ports 258 and 262 may be in the range of, e.g., between about ⅛″ to about ¼″ in some embodiments. The first cannula inlet port 224 may be coupled to a first outlet port (not shown) of the infusion pump system 100, and the second cannula inlet port 226 may be coupled to the second outlet port (not shown) of the infusion pump system 100. The dual-lumen cannula 210 may be formed from two substantially identical cannulas by bending and attaching two cannulas together at their distal portion. The attached portion 215 of the two cannulas may include a configuration without any saddle between the two cannulas 214 and 216.

FIG. 4 shows a perspective view of a dual-lumen cannula embodiment 230 of the infusion set 200 of FIG. 2 according to another embodiment. The dual-lumen cannula 230 may be formed as a monolith cannula with two lumens, a first lumen 234, and a second lumen 236. The first lumen 234 and the second lumen 236 may be disposed inside a generally cylindrical body 232. The first lumen 234 may deliver the first therapeutic fluid 248 and the second lumen 236 may deliver the second therapeutic fluid 252 to their respective outlet ports 238 and 242. Outlet ports 238 and 242 may have an offset 294 in the range of, e.g., between about 1/12″ to about ⅙″.

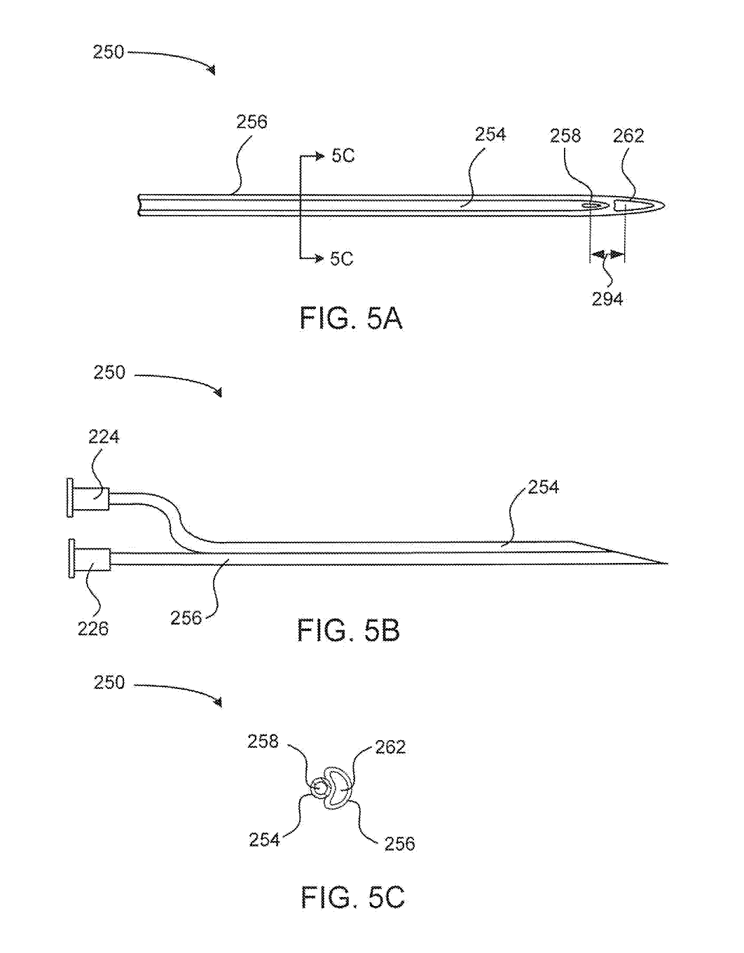

FIGS. 5A-5C show different views of a dual-lumen cannula embodiment 250 of the infusion set 200 of FIG. 2. The dual-lumen cannula 250 may include a first cannula 254 and a second cannula 256. The first cannula 254 may include a first lumen that extends within a length of the first cannula. The first lumen may have a circular cross-section. The second cannula 256 may include a second lumen that extends within a length of the second cannula. The second lumen may have a generally crescent-shaped cross-section. A size of the cross-section of the second lumen may be larger than of the size of a cross-section of the first lumen. The dual-lumen cannula 250 may be formed by joining distal portions of the first cannula 254 and the second cannula 256 and securing this portion together. The distal portion of the first cannula 254 may be fitted into a concave side or a groove of the second cannula 256. Output ports of the first cannula 254 and the second cannula 256 may be offset from each other as indicated by arrow 294 in FIG. 5A. The output port 262 of the second cannula may be positioned adjacent to a distal tip of the dual-lumen cannula 250 in some cases.

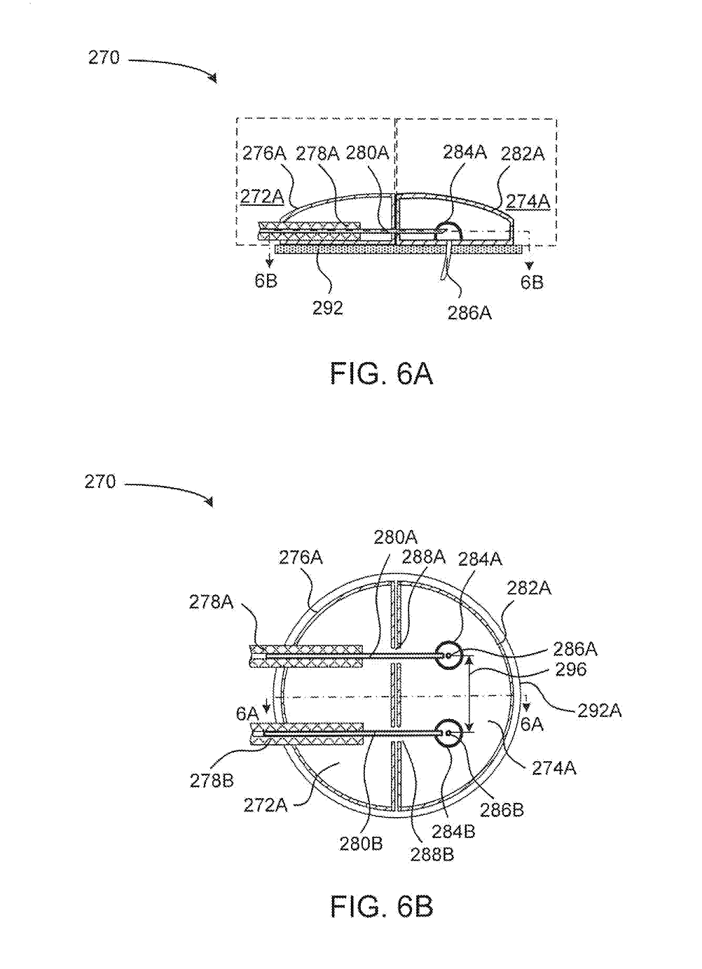

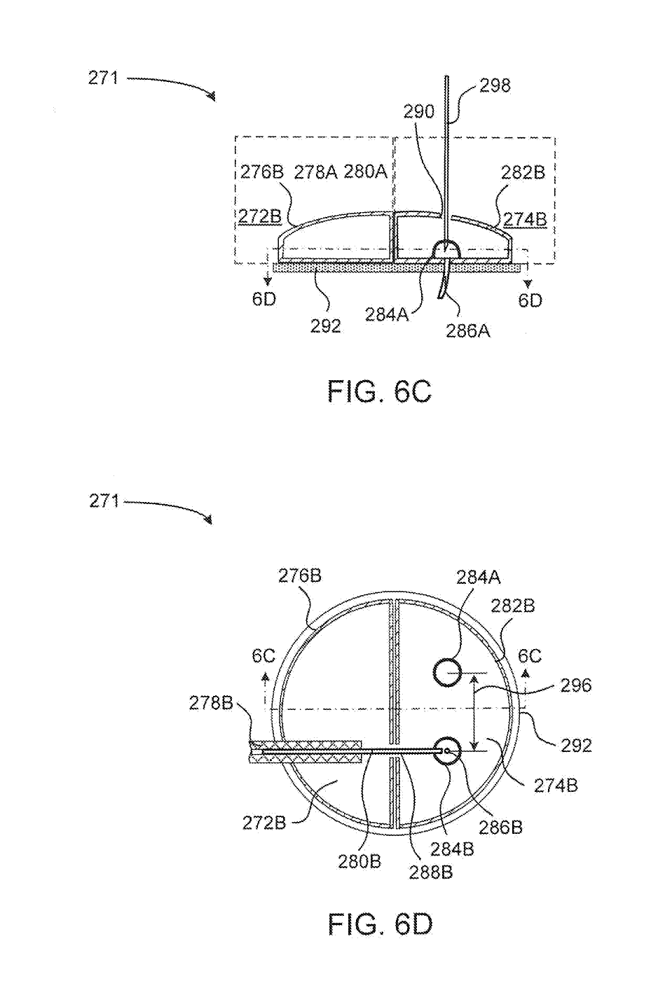

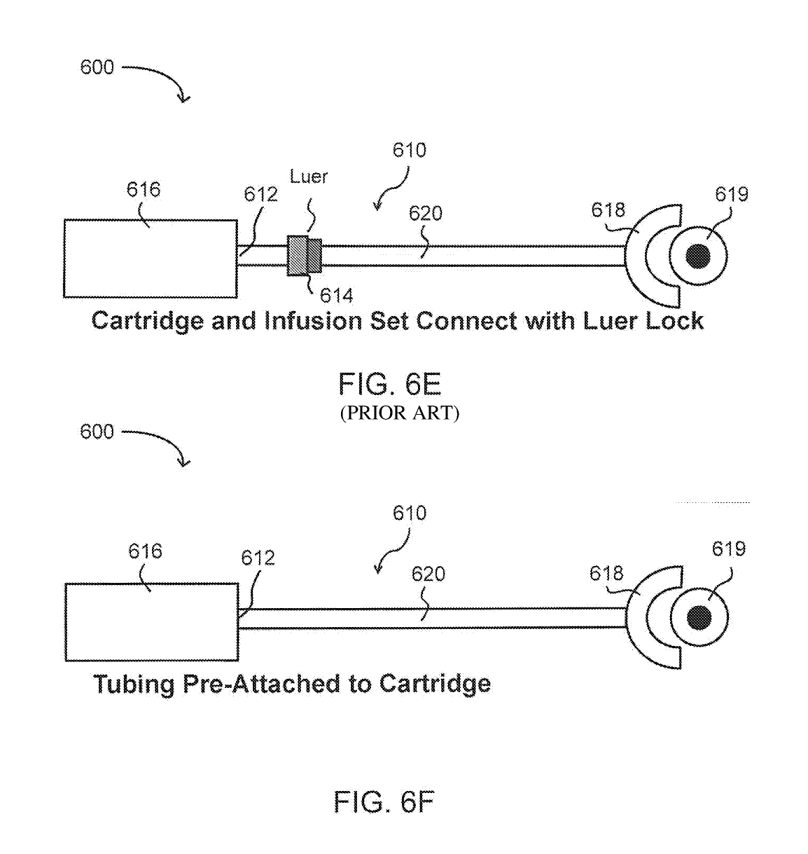

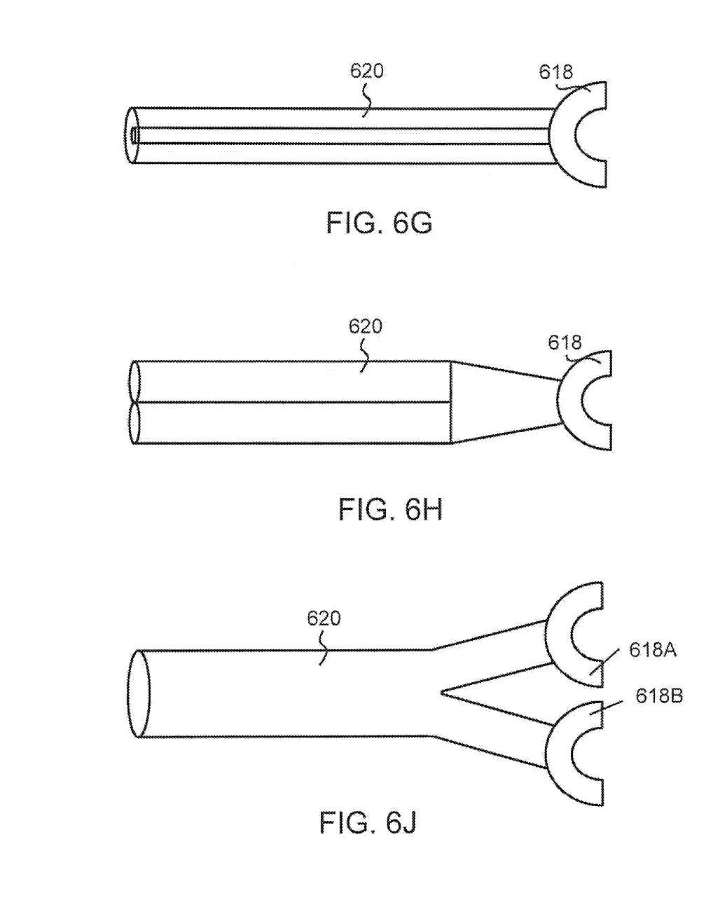

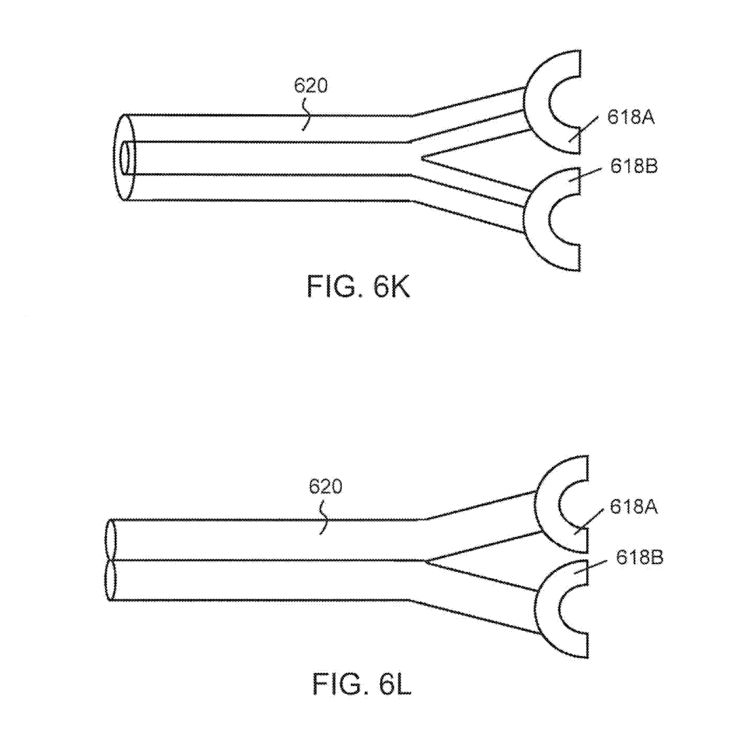

FIGS. 6A and 6B show in cross-section a dual-lumen cannula assembly embodiment 270 of the infusion set 200 of FIG. 2. The dual-lumen cannula assembly 270 may be configured to be attached to a patient's skin 304 with a flexible adhesive layer 292 and to be coupled to the infusion pump system 100.

The dual-lumen cannula assembly 270 may include a cannula housing 274A and a connector assembly 272A. The cannula housing 274A may have a top shell 282A. Two side-access ports 288A and 288B may be provided in the top shell 282A. Two self-sealing septa 284A and 284B may be disposed in a bottom portion of the cannula housing 274A. A catheter 286A or 286B may be disposed under each of the self-sealing septa. A placement distance (offset) 286 between catheters 286A and 286B may be in the range of, e.g., between about ⅛″ to about ¼″ for some embodiments. An offset between the catheters may prevent the mixing of medications which may allow the delivery of multiple medications to the patient which may be incompatible with each other.

The connector assembly 272A may be removably connected to the cannula housing 274A. The connector assembly 272A may include two connecting needles, a first connecting needle 280A, and a second connecting needle 280B. The connecting needles have proximal ends and distal ends. The distal ends of the connecting needles 280A and 280B may be configured to pierce into the self-sealing septa 284A and 284B and to form fluid passageways in fluid communication with lumens of the catheters 286A and 286B respectively. The proximal ends of the connecting needles 280A and 280B may be embedded into the connector assembly inlet ports 278A and 278B respectively. The first and second connector assembly inlet ports 278A and 278B may be attached to a shell 276A of the connector assembly 272A. The first and second connector assembly inlet ports 278A and 278B may be connected to and be in fluid communication with the outlet port of a delivery device, for example, a delivery mechanism of the infusion pump system embodiments described herein via fluid tubing 101 (see, e.g., FIGS. 1-2).

FIGS. 6C and 6D show in cross-section a dual-lumen cannula assembly embodiment 271 of the infusion set 200 of FIG. 2. The dual-lumen cannula assembly 271 may be configured to be attached to a patient's skin 304 with a flexible adhesive layer 292 and to be coupled to the infusion pump system 100. The dual-lumen cannula assembly 271 in most aspects may be similar to the dual-lumen cannula assembly 270. However, for this embodiment, a top shell 282B of a cannula housing 274B may have only one side-access port 288B and may also have a top-access port 290 in the top shell 282B. The top-access port 290 may be configured to allow a single needle 298 to pierce into the self-sealing septum 284A to form fluid passageways to the catheter 286A. A connector assembly 272B may include one connecting needle 280A. The second connector assembly inlet ports 278A and the distal end of the single needle 298 (not shown) may be connected to and be in fluid communication with the outlet port of a delivery device.

FIGS. 6E and 6F show embodiments of infusion kits 600A, 600B. Each infusion kit can include an infusion cartridge 616. The infusion cartridge 616 may be configured to be coupled to an infusion pump assembly such as infusion pump assembly 10 or any other suitable infusion apparatus. The infusion cartridge 616 may include a rigid housing and a collapsible fluid reservoir formed from a flexible material and disposed within the rigid housing. A dispense mechanism may be fluidly connected to the fluid reservoir. The infusion cartridge 616 may have an outlet port or dispense port 612 that may be connected to an infusion set connector 614 and an infusion set 610. In such embodiments, at the other end of the long length of tubing 620 is a connector 618 that may attach or be pre-connected to a cannula and/or infusion needle that punctures the patient's skin at the infusion site. In such cases, the tubing is connected to the cannula with an inner lumen of the tubing 620 in fluid communication with an inner lumen of the cannula or infusion needle.

The infusion kit 600A in FIG. 6E is an infusion kit as is known in the prior art in which tubing connects to an infusion cartridge 616. The infusion set connector 614 includes a short length of tubing 620 and a male Luer lock fitting, as shown in FIG. 6E. The short length of tubing 620 terminates at the male Luer lock and may range in length from about 1 inch to about 10 inches, such as from about 3 inches to about 6 inches. The male Luer lock fitting attaches to a female Luer lock fitting that is at the end of the length of tubing 620. The assembly of the female Luer lock fitting, the long length of tubing, the connector and the cannula may be considered as an infusion set 610.

FIG. 6F depicts an infusion set 610 according to an embodiment of the present invention in which the infusion set 610 can be preattached to the cartridge 616. The infusion set 610 includes a length of tubing 620 that extends from an outlet port of the cartridge and terminates in the connector 618. The tubing can extend from the port to the connector as a solid, uninterrupted unitary piece of tubing with a constant diameter and having no connectors connecting portions of the tubing. In such embodiments, the connector 618 may allow fluid to flow from the infusion cartridge 616, through an inner lumen of the tubing 620 and into the cannula that pierces the patient's skin so as to put an inner lumen of the tubing and an inner lumen of the cannula into fluid communication with the interstitial space of the patient's body or some other suitable portion within the patient's body. In these embodiments, the infusion site 619 includes the cannula and/or infusion needle, and optionally includes an insertion mechanism. In some embodiments the infusion set 610 includes a length of tubing 620 ranging from about 1 inch to about 50 inches, such as from about 2 inches to about 20 inches, or such as from about 3 inches to about 6 inches, including about 4 to about 5 inches.

Notable in these embodiments as shown in FIG. 6F is the absence of a Luer lock or other easily detachable connections or mechanism. A lack of easily detachable connections, such as Luer lock connections and the like, between two lengths of tubing can reduce the likelihood of leakage from the tubing and reduce the number of connections a patient needs to perform. Such a design also improves comfort and reduces the risk of snagging the line on clothing or other objects. In some such embodiments, the infusion set 610 may be pre-attached to the infusion cartridge 616 in a permanent manner such as by adhesive bonding, welding, monolithic or integral formation or the like such that the tubing cannot be removed from the infusion cartridge without damaging the tubing. Such a set can be provided along with the cartridge in a single sterile package

In some embodiments, the infusion cartridge may have an outlet port that may be connected in fluid communication to an infusion set 610 that includes a length of tubing that terminates in a connector. In such embodiments, the length of tubing may have more than one lumen. In such embodiments, the tubing may be capable of delivering more than one fluid to a single infusion site. Tubing 620 that is capable of delivering more than one fluid to a single infusion site may include coaxial lumina, such as a length of tubing within another length of tubing as shown in FIG. 6G, or it may include two or more lengths or sections of tubing side by side which are joined at the terminus before the connector 618 as in FIG. 6H. In some embodiments, the infusion cartridge may contain more than one fluid and have more than one outlet port that may be connected to an infusion set. Such embodiments may include those in which the infusion set 610 is pre-attached to the infusion cartridge 616 as well as those embodiments in which the infusion set 610 is attached by a user to the infusion cartridge 616 using an easily detachable configuration such as a Luer connector.

In some embodiments, the infusion cartridge 616 may have an outlet port 612 that may be connected in fluid communication to an infusion set 610 that includes a length of tubing 620 that terminates in more than one connector 618A and 618B. In such embodiments, the tubing 620 is capable of delivering fluid to more than one infusion site. Embodiments of tubing 620 that are capable of delivering fluid to more than one infusion site may include a single length of tubing that is split into two lengths of tubing, such that it resembles the letter “y” as in FIG. 6J. Such “y” shaped tubing configurations may also include tubing that is capable of delivering more than one fluid to a single infusion site, such that the tubing includes lengths or sections of coaxial lumina as shown in FIG. 6K. Alternatively, tubing 620 that is capable of delivering fluid to more than one infusion site may include two lengths or sections of tubing that are fused or joined together until near the terminus where the tubing 620 is connected to two or more connectors 618A and 618B, such as shown in FIG. 6L. The “y” shaped tubing configurations described herein may be pre-attached in a permanent or substantially permanent manner to the infusion cartridge 616 or may be attached to the infusion cartridge 616 by a user as needed.

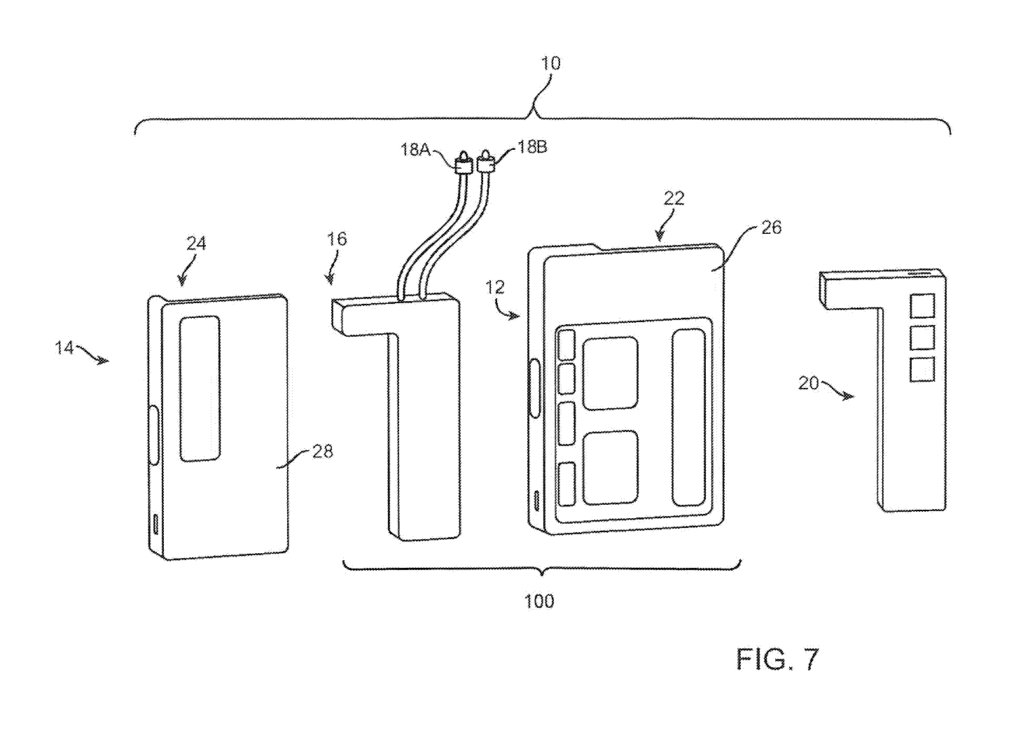

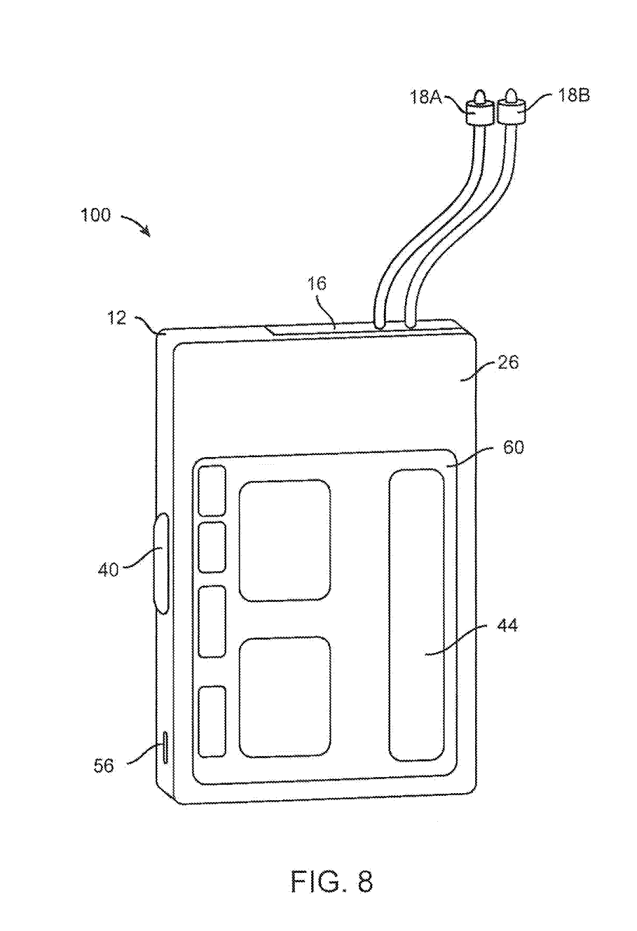

FIG. 7 shows an embodiment of an interchangeable infusion pump assembly 10 that may be useful as fluid delivery device. The infusion pump assembly 10 may be configured to deliver a fluid or fluids from one or more reservoirs which are in fluid communication with a delivery mechanism thereof. The infusion pump assembly 10 may include a pump 12, a second pump 14, an infusion cartridge 16 having infusion set connectors 18A and 18B, and optionally a glucose meter 20. The infusion cartridge may include multiple collapsible fluid reservoirs in fluid communication with respective inlet ports as shown in the embodiments of FIG. 13A and discussed in the text below. Either the infusion cartridge 16 or the glucose meter 20 may be functionally and interchangeably inserted in a first receiving slot 22 located in the pump 12 and a second receiving slot 24 located in the second pump 14.

The pump 12 may have a housing 26 that may be generally larger than a second housing 28 of the second pump 14. Similarly, the pump 12 generally may include more operating features than the second pump 14. Some or all of the suitable features, dimensions, materials, and methods of use of the infusion pump assembly 10 shown in FIG. 7 may be used or incorporated into any other infusion system, or components thereof, discussed herein. It should also be noted that the interchangeability of infusion cartridge embodiments is discussed herein generally in the context of transferring an infusion cartridge from the pump 12 the second pump 14 having features different from those of the pump 12. However, all of the interchangeability features and methods associated with this type of transfer may also be applied to the transfer of an infusion cartridge from the pump 12 to the second pump 14 having the same features as the pump 12.

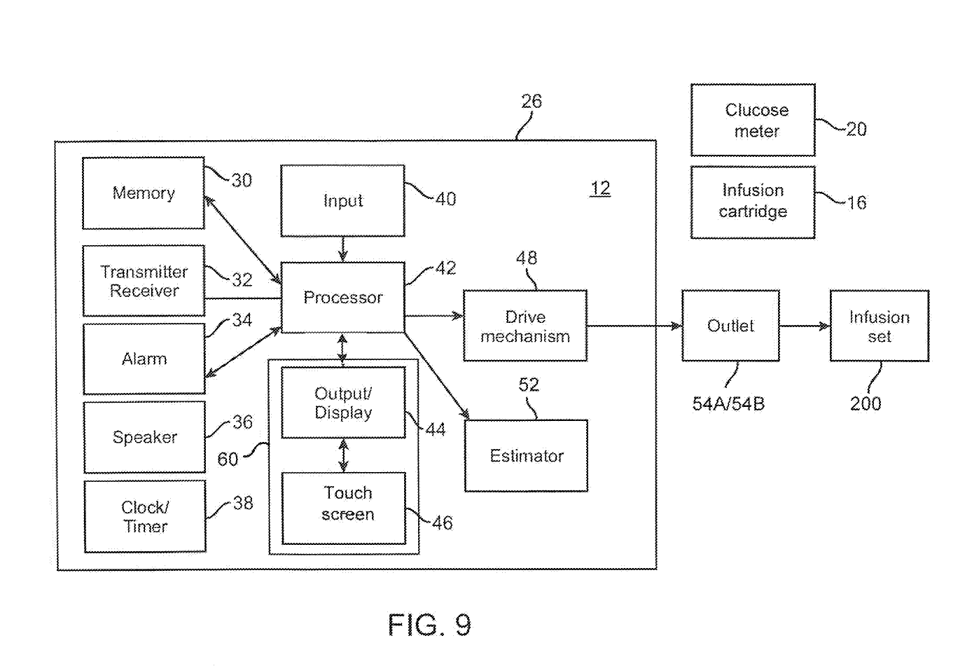

FIGS. 8 and 9 show an embodiment of a pump system 100. The pump system 100 may include the pump 12 and the infusion cartridge 16. The housing 26 of the pump 12 can be of any suitable shape and size. For instance, the housing 26 may be extended and tubular, or in the shape of a square, rectangle, circle, cylinder or the like. The housing 26 may be dimensioned so as to be comfortably associated with a user and/or hidden from view, for instance, within the clothes of a user. In some embodiments, the housing 26 of the pump 12 may have a width of about 2 inches to about 5 inches, a height of about 1 inch to about 3 inches and a thickness of about 0.25 inch to about 0.75 inch, more specifically, the housing 26 may have a width of about 2.5 inches to about 3.5 inches, a height of about 1.5 inches to about 2.5 inches and a thickness of about 0.4 inches to about 0.8 inches. The materials of the housing 26 may vary as well. In some embodiments, housing of the pump 12 may be a watertight, metal housing that may be taken apart for repairs.

The pump 12 may include a graphic user interface (GUI) 60. The GUI 60 may include an output/display 44. The output/display 44 may vary, as it may be useful for a particular application. The type of visual output/display may include LCD displays, LED displays, plasma displays, OLED displays, and the like. The output/display may also be an interactive or touch sensitive screen having an input, such as, e.g., a touch screen such as a capacitive screen, a resistive screen, or the like. In some embodiments, the output/display 44 of the pump 12 may be an OLED screen and the input may be a capacitance touch screen. The pump 12 may additionally include a keyboard or another input 40 known in the art for data entry, which may be separate from the display. The output/display 44 of the pump 12 may also include a capability to operatively couple to a secondary display device such as a laptop computer, a mobile communication device, such as, e.g., a smartphone or personal digital assistant (PDA), or the like.

The pump 12 may have wired or wireless communication capability for the sending and receiving of data as is known in the art. The wireless capability may be used for a variety purposes, including updating of any software or firmware for the processor of the device. The wireless communication capability may vary including, e.g., a transmitter and/or receiver, radiofrequency (RF) transceiver, WIFI connection, infrared or Bluetooth® communication device. The wired communication capability may also vary including, e.g., USB or SD port, flash drive port, or the like. In some embodiments, the pump 12 may have a transmitter/receiver 32, such as a radiofrequency (RF) transceiver as shown in FIG. 9 that may allow the more than one pump 12 to communicate with one another and may be used interchangeably without loss of data or information during an infusion protocol with an infusion cartridge 16. The pump 12 may also act as a PDA or controller that may wirelessly control the second pump 14 as shown in FIG. 7. For such an embodiment, data may be transferred between the controller of the pump 12 and the second pump 14 by radio signal, optical transmission or any other suitable means. The pump 12 and the second pump 14 may be used as stand-alone devices as well.

One or more of the pumps may also include GPS functionality, phone functionality, warning and/or alarm programming; music storage and replay functionality, e.g., an MP3 player; a camera or video mechanism; auto scaling capabilities, and/or one or more video type games or other applications developed by third parties for use thereon. One or more of the pumps may also include an accelerometer, for instance, which may be used for changing presented estimates, wherein instead of scrolling through a menu of options or using a numerical keypad, values can be input or changed via the accelerometer, such as by gesturing with or otherwise shaking the pump.

The pump 12 may include a memory device 30. The memory device 30 may be any type of memory capable of storing data and communicating that data to one or more other components of the device, such as the processor. The memory may be one or more of a Flash memory, SRAM, ROM, DRAM, RAM, EPROM, dynamic storage, and the like. For instance, the memory may be coupled to the processor and configured to receive and store input data and/or store one or more template or generated delivery patterns. For example, the memory can be configured to store one or more personalized (e.g., user defined) delivery profiles, such as a profile based on a user's selection and/or grouping of various input factors, for example, past generated delivery profiles, recommended delivery profiles, one or more traditional delivery profiles, e.g., square wave, dual square wave, basal profiles, bolus rate profiles, and/or the like. The memory can also store user information, for example, history of use, glucose measurements, compliance, an accessible calendar of events, and the like. In some embodiments, the memory 30 of the pump 12 may be up to about 10 GB, more specifically, up to about 3 GB, even more specifically, about 1 MB to about 200 MB. In some embodiments, the memory 30 of the pump 12 may be up to about 3 GB, more specifically, up to about 500 MB, and even more specifically, about 200 kB to about 200 MB.

The pump 12 may include a power charging mechanism in some cases, such as a USB port, induction charger, or the like. The power charging system may be used to charge a power storage cell such as a rechargeable battery of the pump 12. Some embodiments may use a rechargeable battery such as a NiCad battery, LiPo battery, NiMH battery or the like. In some embodiments, the power charging mechanism 56 of the pump 12 shown in FIG. 8 may be a USB port. As such, all data may be kept in the pump 12 for quick and easy downloading of data to a computer, other pump, network etc. using the USB port. The USB port 56 of the pump 12 may also provide the pump 12 with power charging. In some instances, the power charging mechanism of the pump 12 may be an induction-charging device. In some cases, an advantage of having interchangeable pumps may be that while one pump is being used for infusion, the other pump can be charging. Further, the use of dual pump may provide a user of the pump with a backup in case of failure of one pump.

The pump 12 may also include programming to allow a processor 42 to make a recommendation regarding a variety of treatment parameters. For instance, the processor 42 may include one or more estimator functionalities/devices 52, which may allow the processor 42 to receive data from various sources, parse the data, collate the same, and generate an estimate based on the same. For instance, the processor 42 may receive user input data and/or data from one or more sensors or other external sources, which the processor 42 can process and thereby use to generate an estimate, such as an estimate of an amount of fluid to be delivered to a body, a rate of fluid delivery, and/or a specific fluid delivery profile. For example, the processor 42 may be configured to process data pertinent to a current or predicted condition and to generate an estimate, represented as an amount, rate, profile, etc. of fluid to be delivered based on that data, which estimate may then be displayed to a user, thereby allowing the user to interact with the estimate to accept, decline, and/or otherwise modify the estimate. The processor 42 may include programming that functions to control the respective device and its components. The processor 42 may communicate with and/or otherwise control the drive mechanism, output/display, memory, a transmitter/receiver, and the like. The processor of one of the pumps may communicate with the processor of the other pump, for example, through the transmitter/receiver. The processors may include programming that can be run to control the infusion of insulin or other medicament from the cartridge, the data to be displayed by the display, the data to be transmitted via the transmitter, etc. The processors may also include programming that may allow the processors to receive signals and/or other data from an input device, such as a sensor that senses pressure, temperature, and the like, that may be included as a part of the pump or may be used in conjunction therewith. The processor 42 may receive signals, for instance, from the transmitter/receiver that may be part of the blood glucose monitor and store the signals in the memory.

FIG. 9 is a block diagram illustrating some of the features/components that may be incorporated within the housing 26 of the pump 12. The pump 12 can include a memory device 30, the transmitter/receiver 32, an alarm 34, a speaker 36, a clock/timer 38, the input device 40, the processor 42, the GUI 60 having a touch screen 46 with input capability, a drive mechanism 48, and the estimator device 52. As mentioned, the housing 26 of the pump 12 may be functionally associated with an interchangeable and a removable glucose meter 20 and/or infusion cartridge 16. The infusion cartridge 16 may have outlet ports 54A and 54B that may be connected to the infusion set 200 via infusion set connectors 18A and 18B respectively (see FIG. 7).

The processor 42 may also include additional programming to allow the processor 42 to learn user preferences and/or user characteristics and/or user history data, for instance, to implement changes in use, suggestions based on detected trends, such as, e.g., weight gain or loss; and may include programming that allows the device to generate reports, such as reports based upon user history, compliance, trending, and/or other such data. Additionally, pump device embodiments of the disclosure may include a “power off” or “suspend” function for suspending one or more functions of the device, such as, e.g., suspending a delivery protocol, and/or for powering off the device or the delivery mechanism thereof. For some embodiments, two or more processors may be used for controller functions of the pumps, including a high power controller and a low power controller used to maintain programming and pumping functions in low power mode in order to save battery life.

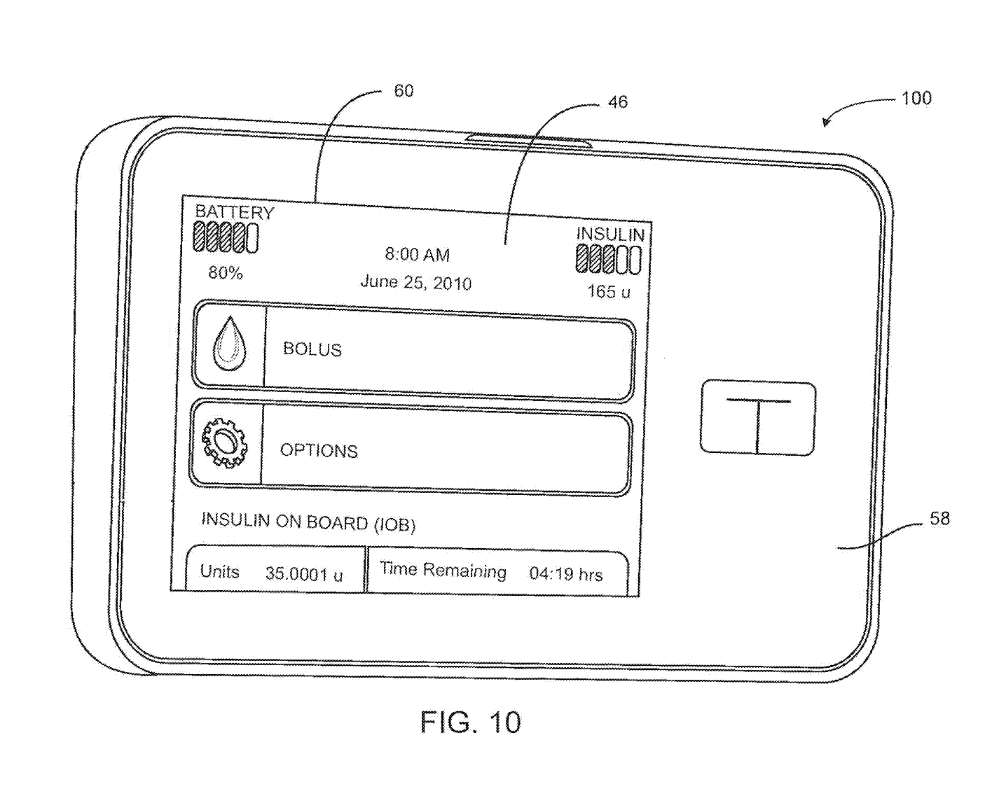

FIG. 10 shows a front view of the infusion pump system 100. The infusion pump system 100 may include a user-friendly graphic user interface 60 on a front surface 58 of the infusion pump system 100. The GUI 60 may include the touch sensitive screen 46 that may be configured to display a variety of screens used for displaying data, facilitating data entry by a patient, providing visual tutorials, as well as other interface features that may be useful to the patient 300 operating the infusion pump system 100.

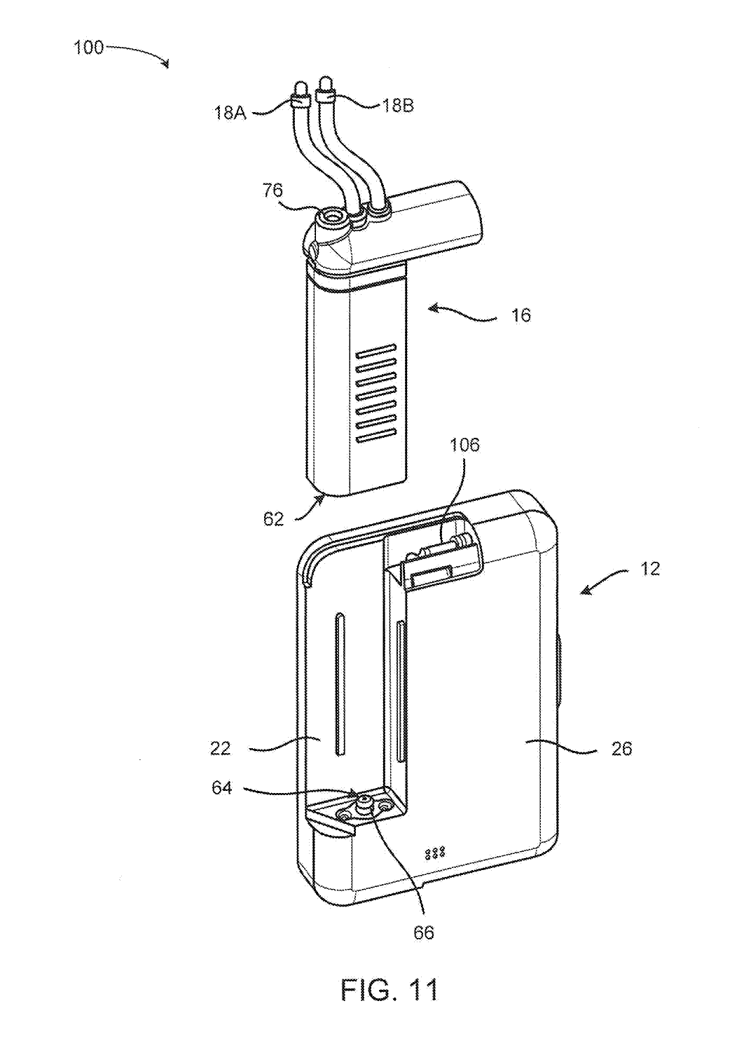

FIG. 11 shows the infusion pump system 100 with the infusion cartridge 16 detached from the pump 12, the pump 12 may include an attachment mechanism 64 positioned within the first receiving slot 22 near its terminus that corresponds to a receiving mechanism 62 at an end of the infusion cartridge 16. The attachment and receiving mechanisms may be configured to removably couple an interior volume of the cartridge with a volume of the pump that may be sealed from the surrounding environment with the coupling able to retain a fluid within the volumes even under significant pressure. The O-ring based tap attachment embodiment discussed below may be so configured and suitable for producing a leak free detachable coupling that can withstand significant pressure. The receiving mechanism 62 may be configured to detachably couple with the attachment mechanism 64 such that the infusion cartridge 16. The infusion cartridge 16 may be reversibly attached to the housing 26 of the pump 12 for fluid delivery. In this embodiment, the attachment mechanism 64 may include a pneumatic tap 66 having an O-ring or other sealing device. The corresponding receiving mechanism 62 positioned on an end of the infusion cartridge 16 may include a port through which the pneumatic tap 66 may be inserted. A reservoir fill port 76 may be disposed on a top portion of the infusion cartridge 16. In some cases, if the desired fluid may be manually dispensed from the interior volume of a syringe, through the reservoir fill port 76 into the interior volume of the infusion cartridge 16.

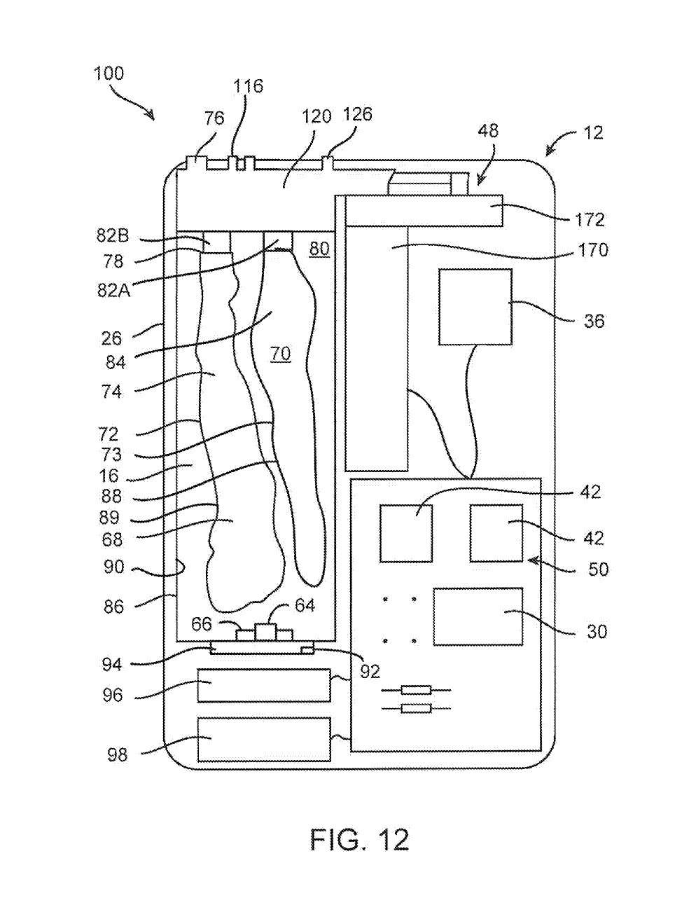

Referring to FIG. 12, two collapsible fluid reservoirs 70 and 68 of the infusion cartridge 16 may be bounded by or disposed within flexible membranes or layers 72 and 73 respectively. The first collapsible fluid reservoir 70 may include an interior volume 84 in fluid communication with a first inlet port 108 of an axial bore 104 of a delivery mechanism 120. A top portion of the first collapsible fluid reservoir 70 may be clamped or otherwise sealed to an extension or a boss 82A of the first inlet port 108 that extends into a first vented volume 80. In this configuration, the interior volume 84 of the first collapsible fluid reservoir 70 may be isolated or sealed from the surrounding environment except for the first inlet port 108 which may be in fluid communication with the axial bore 104 of the delivery mechanism 120.

A second collapsible fluid reservoir 68 may include an interior volume 74 in fluid communication with a second inlet port 112 of an axial bore 104 of a delivery mechanism 120. A top portion of the second collapsible fluid reservoir 68 may be clamped or otherwise sealed to an extension or a boss 82B of the second inlet port 112 that extends into a first vented volume 80. In this configuration, the interior volume 74 of the collapsible fluid reservoir 68 may be isolated or sealed from the surrounding environment except for the second inlet port 112 which is in fluid communication with the axial bore 104 of the delivery mechanism 120.

A substantially rigid shell 86 may be disposed about the first and the second collapsible fluid reservoirs 70 and 68 with a first vented volume 80 that may contain the collapsible first and the second collapsible fluid reservoirs 70 and 68. The first vented volume 80 of the infusion cartridge 16 may be disposed between the outer surfaces 88 and 89 of the flexible membranes 72 and 73 and an interior surface 90 of the rigid shell 86. A vent inlet port 124 may be in fluid communication with the first vented volume 80 and the axial bore 104 of the delivery mechanism 120.

As shown in FIG. 12, a controller 50 may be operatively coupled to an electric motor 170. The controller 50 may include at least one processor 42, a memory device 30 and connective circuitry or other data conduits that couple the data generating or data managing components of the device. A power storage cell in the form of a battery 98 that may be rechargeable may also be disposed within the housing 26. Data generating or managing components of the device may include the processor(s) 42, the memory device 30, and sensors, including any pressure or temperature sensors, the GUI 60 (see FIG. 9), and the like. Other components such as a vibratory motor 96, the speaker 36, and the battery 98 may also be operatively coupled to the controller 50. Connective circuitry may include conductive wiring such as copper wiring, fiber optic conduits, RF conduits and the like. For some embodiments, the infusion cartridge 16, and any of the collapsible fluid reservoir cartridges discussed herein, may include an encoder or bar code type strip (not shown). The encoder strip or device may be configured to be scanned and read by a reader device of the infusion pump system 100, the reader device in operative communication with the controller 50 or processor 42 thereof. The encoder device may alternatively be an RFID chip or the like that transmits data to a reader such as a data receiving processor or the like. Such encoder device embodiments may include the ability to securely transmit and store data via, encryption, to prevent unauthorized access or tampering with such data. The identification of the infusion cartridge 16 may be used by the controller 50 to set or to adjust certain dispense parameters or any other suitable parameter.

The vent inlet port 124 may be disposed on the delivery mechanism 120 in fluid communication with the first vented volume 80 disposed between the outside surfaces 88 89 of the flexible materials or membranes 72 and 73 of the collapsible fluid reservoirs 68, 70, and the inside surface 90 of the substantially rigid shell or case 86 of the infusion cartridge 16. The controller 50 may be coupled to at least one pressure sensor 92 which may be disposed in communication with a chamber 94 which may be in communication with the first vented volume 80 by means of attachment mechanism 64 and receiving mechanism 62. The controller 50 may be configured to generate signal to the drive mechanism 48 to displace the spool 106 of the delivery mechanism 120.

FIG. 13 shows a portion of the infusion cartridge 16 including the delivery mechanism 120 as well as a portion of the drive mechanism 48 of an infusion pump system 100. The delivery mechanism 120 may be configured to deliver material from the first and the second collapsible fluid reservoirs 70 and 68 via a collapsible or variable volume element of a spool 106. For the embodiments discussed herein, the variable volume elements may include constrained variable volume elements that are mechanically constrained to vary between a minimum volume and a maximum volume. The delivery mechanism 120 may include a delivery mechanism body 102, or housing, and an axial bore 104 disposed in the delivery mechanism body 102. The axial bore 104 may have a substantially round transverse cross-section. The spool 106 may also have a substantially round transverse cross-section, and may be slidingly disposed within the axial bore 104. The drive mechanism 48 includes a rack and pinion mechanism 174 actuated by an electric motor 170 through a gear box 172.

For some embodiments, the axial bore 104 of the delivery mechanism may have a transverse dimension or diameter of about 0.04 inches to about 0.5 inches, more specifically, about 0.08 inches to about 0.15 inches. For some embodiments, the spool 106 may have a length of about 10 mm to about 40 mm, more specifically, about 15 mm to about 20 mm. The spool 106 and housing of the delivery mechanism 120 may be made from any suitable material or materials including polymers or plastics such as polycarbonate, PEEK, thermoplastics, cyclic olefin copolymer, and the like. In some cases, the seals disposed on the spool 106 may have an outer transverse dimension or diameter that may be slightly larger than that of the spool 106. In some instances, the seals on the spool 106 may have an axial thickness of about 0.01 inches to about 0.03 inches and may be made from materials such as butyl, silicone, polyurethanes or the like having a shore hardness of about 65 A to about 75 A, more specifically, about 70 A.

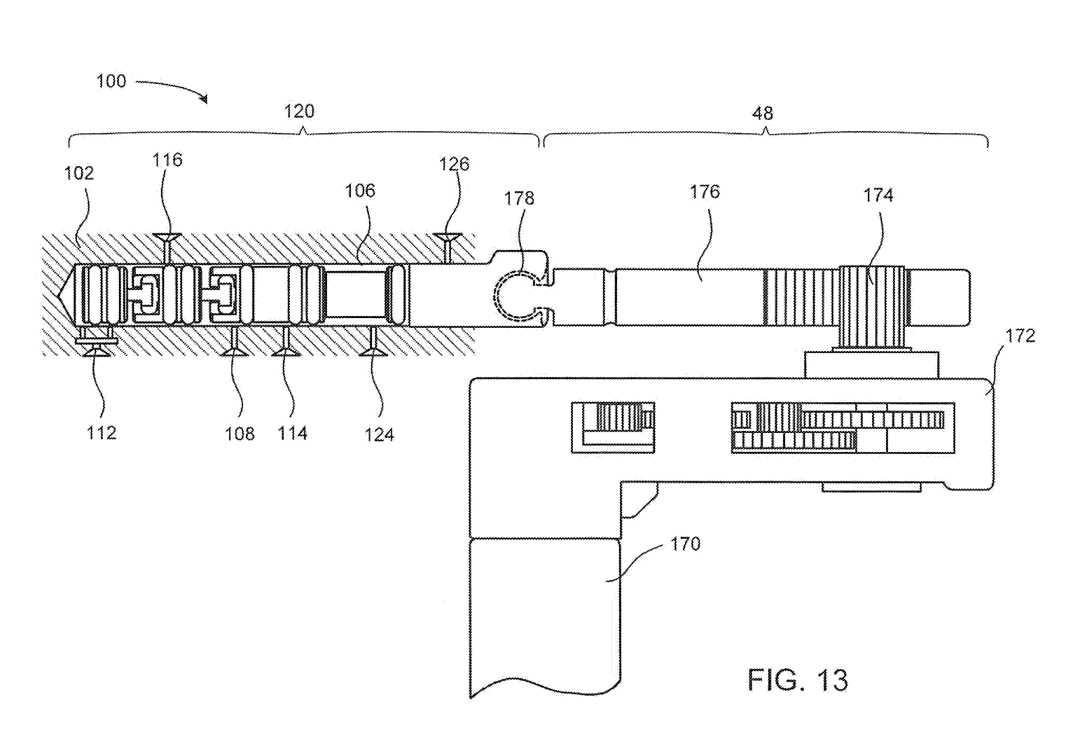

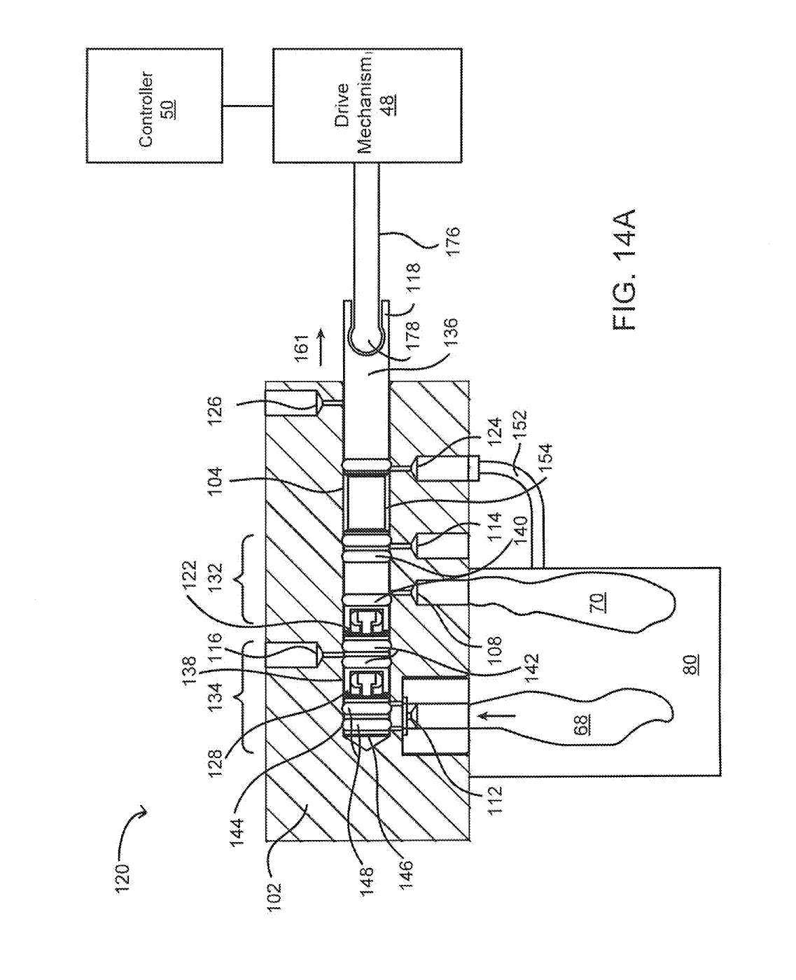

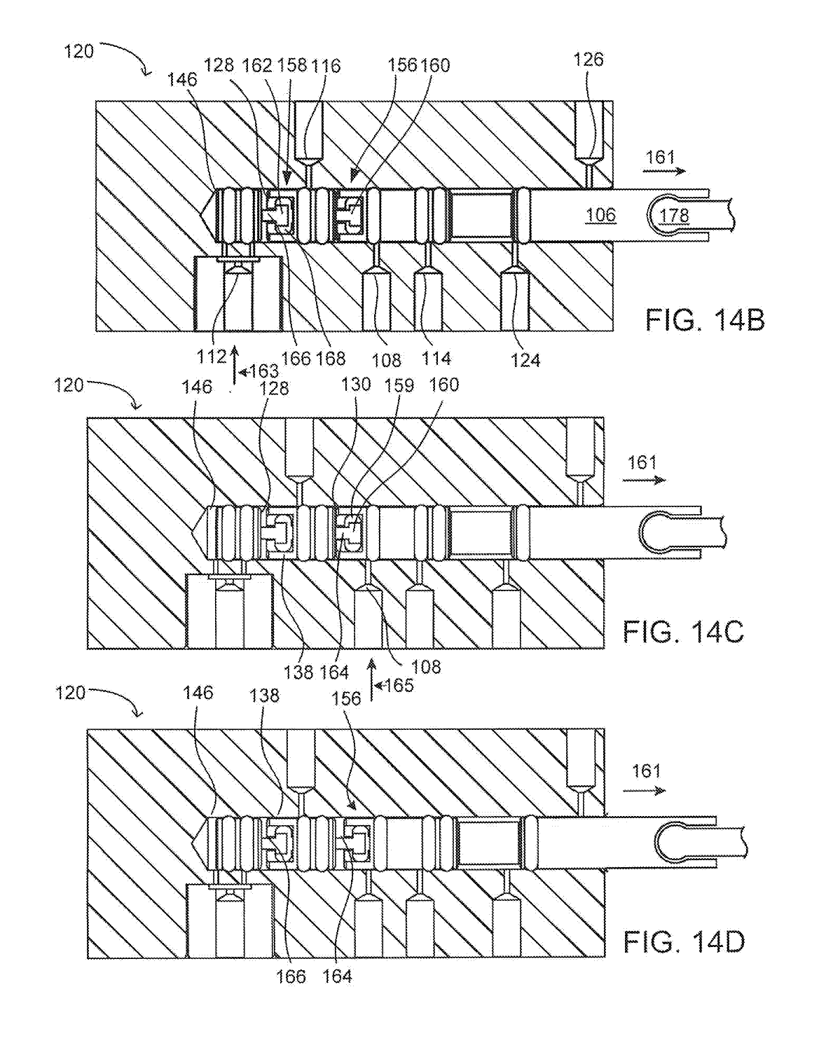

FIG. 14A illustrates a delivery mechanism 120 that is configured to deliver two separate fluids from two separate collapsible fluid reservoirs to two separate respective outlet ports without mixing the two fluids. In some cases, the two outlet ports 114 and 116 may be combined into a single conduit where the fluids would be mixed if such single fluid tubing might be desired. As discussed above, the use of a single spool 106 and the drive mechanism 48 may be useful with regard to the cost of the device, efficient use of stored energy or battery life and overall size and weight of the device. The drive mechanism 48 used in some cases for the delivery mechanism 120 in an embodiment of FIG. 14A may be the same as or similar to the drive mechanism 48 shown in FIG. 12 and discussed above. As such, a proximal end of the spool 106 of the delivery mechanism 120 in FIG. 14A, may include a coupling, such as, e.g., a ball coupling 178, that may be suitable for releasable connection to a distal end of a drive shaft, such as, e.g., drive shaft 176 shown in the embodiment of FIG. 12. The delivery mechanism 120 of FIG. 14A may also have other features, dimensions, modes of operation or materials which are, the same as, or similar to those of the delivery mechanism 120 shown in FIG. 12. In some cases, the spool 106 may be actuated by the drive mechanism 48 including the electric motor 170 powered by a power storage cell 98 and controlled by the controller 50.

In general, an infusion pump embodiment as shown in FIG. 14A may include a first inlet port 108, a first outlet port 114, a first collapsible fluid reservoir 70 in fluid communication with the first inlet port 108, a second inlet port 112, a second outlet port 116 and the second collapsible fluid reservoir 68 in fluid communication with the second inlet port 112. A first constrained variable volume 122 may be translatable between a position in fluid communication with the first inlet port 108 and a position in fluid communication with the first outlet port 114. A second constrained variable volume 128 may be translatable between a position in fluid communication with the second inlet port 112 and a position in fluid communication with the second outlet port 116. In some cases, the drive mechanism 48 may be configured to expand or contract the first constrained variable volume 122 and the second constrained variable volume 128 due to exertion of a translational force through a boundary section of the respective constrained variable volumes 122 and 128.

More specifically, the delivery mechanism embodiment 120 shown in FIG. 14A includes the axial bore 104. The axial bore 104 may have a first delivery section 132, a second delivery section 134 and a spool 106 disposed in the axial bore 104. The first delivery section 132 may include the first inlet port 108 and the first outlet port 114, with said first inlet port 108 and the first outlet port 114 being in fluid communication with an interior volume of the first delivery section 132. The second delivery section 134 may include the second inlet port 112 and second outlet port 116, the second inlet port 112 and second outlet port 116 being in fluid communication with an interior volume of the second delivery section 134.

The spool 106 may be axially translatable within the axial bore 104 and have a first spool section 136 including a proximal end 118 configured to couple to a drive shaft 176 of a drive mechanism, such as, e.g., the drive mechanism 48 shown in FIGS. 9, 12 and 14A above. The first spool section 136 also has a first seal 140 which forms a fluid tight seal between the first spool section 136 and an interior surface of the axial bore 104. The first seal 140 or elements thereof may be disposed in a gland or glands of the first spool section 136 in some cases so as to be substantially axially fixed relative to the first spool section 136. The first seal 140 of the first spool section 136 may include a double element seal, the seal may include two O-ring type seal elements 144 that are axially spaced from each other. The double seal elements of the first seal 140 may provide stability for the first spool section 136 as well as a slidable seal arrangement with respect to the axial bore. An outer surface of the first seal 140 may be slidable relative to the interior surface of the axial bore 104 to allow the first spool section 136 to move axially within the axial bore 104 while maintaining a fluid tight seal therewith.

The spool 106 may also include a second spool section 138 having a proximal end coupled to a distal end of the first spool section 136 by a first limited displacement coupling 156. The second spool section 138 may include a second seal 142 which may form a fluid tight seal between the second spool section 138 and the interior surface of the axial bore 104. The second seal 142 may also be substantially axially fixed relative to the second spool section 138 and slidable relative to the interior surface of the axial bore 104 same as the first seal 140. The second seal 142 may also include a double seal arrangement with a double seal element, the seal arrangement may include two O-ring type seals 144 elements which may be axially spaced from each other. The spool 106 may also have a third spool section 146 which may have its proximal end coupled to a distal end of the second spool section 138 by the first limited displacement coupling 156. The third spool section 146 may also have a third seal 148 which forms a fluid tight seal between the third spool section 146 and the interior surface of the axial bore 104. The third seal 148 may be substantially axially fixed relative to the third spool section 146 and axially slidable relative to the interior surface of the axial bore 104 same as the first seal 140. The third seal 148 also includes a double seal arrangement with a double seal element that may include two O-ring type seals element 144 which may be axially spaced from each other.

The first constrained variable volume 122 may be formed between the first spool section 136, the second spool section 138, the first seal 140, the second seal 142, and the interior surface of the axial bore 104. In such a configuration, the first and the second seals 140 and 142 may be axially translatable relative to each other, however mechanically constrained to a maximum and minimum axial separation over a limited axial distance by the first limited displacement coupling 156 that may be operatively coupled between the first spool section 136 and the second spool section 138. The second constrained variable volume 128 may be formed between the second spool section 138, the third spool section 146, the second seal 142, the third seal 148, and the interior surface of the axial bore 104. With this configuration, the second and third seals 142 and 148 are axially translatable relative to each other but mechanically constrained to a maximum and minimum axial separation over a limited axial distance by the first limited displacement coupling 156 operatively coupled between the second spool section 138 and third spool section 146.

The infusion pump includes the first collapsible fluid reservoir 70 having an interior volume 84 in fluid communication with the first inlet port 108 and the second collapsible fluid reservoir 68 having an interior volume 74 in fluid communication with the second inlet port 112. The first collapsible fluid reservoir 70 and the second collapsible fluid reservoir 68, embodiments shown in FIG. 14A, include collapsible reservoirs bounded by a thin flexible fluid tight layers 72 and 73. This configuration may allow the contents of the second collapsible fluid reservoir 68 and the first collapsible fluid reservoir 70 to be withdrawn without the need to vent the first vented volume 80 of the reservoir itself. However, in some cases, the substantially rigid fluid tight shell 86 may be disposed about the first and the second collapsible fluid reservoirs 70 and 68 with the fluid tight first vented volume 80 being formed between an inside surface of the rigid shell 86 and respective outside surfaces of the first and the second collapsible fluid reservoirs 70 and 68. In some circumstances, an interior volume of the rigid shell 86 may form the first vented volume 80 which may allow the volume between the outer surfaces of the first and second collapsible fluid reservoirs 70 and 68 and an inner surface of the rigid shell 86 to accommodate changes in volume of the reservoirs as fluid is withdrawn. For such embodiments, a vent inlet port 124 may be disposed in fluid communication with the interior volume of the axial bore 104 and an interior volume of the first vented volume 80. A vent outlet port 126 may be in fluid communication with the first vented volume 80 of the axial bore 104 and the ambient atmosphere. The spool 106 may include a pair of seals to form a second vented volume 154 such that the first vented volume 80 of the substantially rigid shell 86 may be vented through a conduit 160 in communication with the first vented volume 80 and the axial bore 104 and out into the ambient atmosphere through the vent outlet port 126.

As discussed above, the first and second collapsible fluid reservoirs 70 and 68 may be disposed in the substantially rigid shell 86 having an interior volume that is the first vented volume 80. When venting of the first vented volume 80 is desirable, the controller 50 may be configured to deliver a drive signal to the drive mechanism 48 so as to axially translate the spool 106 to a position whereby the second vented volume 154 of the spool 106 may be in fluid communication with the vent inlet port 124 and the vent outlet port 126. In this position, the first vented volume 80 may be put into fluid communication with the ambient atmosphere or any other desirable or predetermined environment. In addition, a pressure sensor 92 may be disposed within the first vented volume 80 or in fluid communication with the first vented volume 80 to determine the pressure within the first vented volume 80. Such pressure measurements may be useful for determining the amount of fluid dispensed from the first and the second collapsible fluid reservoirs 70 and 68, determining when venting of the first vented volume 80 to be done, detecting leaks or clogs in the infusion pump system or the like.

In some instances, the drive mechanism 48 may be operatively coupled to the proximal end of the first spool section 136. In some of these embodiments, the drive mechanism 48 may be configured to axially translate the first constrained variable volume 122, between the first inlet port 108 and first outlet port 114, and configured to expand the first constrained variable volume 122 while in fluid communication with the first inlet port 108 and contract the first constrained variable volume 122 while in fluid communication with the first outlet port 114. The drive mechanism 48 may also be configured to axially translate the second constrained variable volume 128 between the second inlet port 112 and second outlet port 116 and configured to expand the second constrained variable volume 128 while in fluid communication with the second inlet port 112 and contract the second constrained variable volume 128 while in fluid communication with the second outlet port 116.

The second and first constrained variable volumes 122 and 128 may be expanded or contracted by exertion of axial force through a boundary section of each respective constrained variable volume. In particular, when the drive mechanism 48 imparting axial force on the proximal section of the first spool section 136, that force may be exerted against the contents of the first constrained variable volume 122 by the distal end of the first spool section 136 and the first seal 140. The distal end of the first spool section 136 and the first seal 140 on the first spool section 136 form a boundary section of the first constrained variable volume 122. Axial force from the drive mechanism 48 may be applied to the contents of the constrained variable volume 122. Axial force may result to a proximal portion of the second spool section 138 by increased pressure of the contents of the first constrained variable volume 122 pushing against the proximal end of the second spool section 138 and the second seal 142, which also may be considered to form a boundary section of the first constrained variable volume 122. If the first constrained variable volume 122 is in fluid communication with the first inlet port 108 or the first outlet port 114, the increased or decreased pressure resulting from axial force being applied to the first spool section 136, by the drive mechanism 48, may cause the first constrained variable volume 122 to expand or contract. Such an action may thus draw fluid into the expanding first constrained variable volume 122 or deliver fluid from a contracting first constrained variable volume 122. The same result may occur for the second constrained variable volume 128, except that the axial force applied to the second constrained variable volume 128 is transmitted by the second spool section 138 to the third spool section 146 through a boundary section of the second constrained variable volume 128. In the case of the second constrained variable volume 128 the boundary section may include the distal end of the second spool section 138 and the second seal 142 on the second spool section 138.

In some cases, the drive mechanism 48 and spool 106 may be configured to translate the spool 106 without the first constrained variable volume 122 overlapping the second delivery section 134, or the second constrained variable volume 128 overlapping the first delivery section 132. This type of arrangement may be useful in preventing the mixing of fluids, which are being dispensed from the respective collapsible fluid reservoirs. In some instances, the first delivery section 132 may include a plurality of first inlet ports 108 in fluid communication with the first collapsible fluid reservoir 70. The delivery mechanism 120 may also be configured such that the first constrained variable volume 122 is positionable to overlap the first inlet port 108, the first collapsible fluid reservoir 70 being independent of an overlap with an inlet port in fluid communication with another collapsible fluid reservoir. Likewise, the second delivery section 134 may include a plurality of second inlet ports 112 in fluid communication with the second collapsible fluid reservoir 68, and the delivery mechanism 120, may be configured such that the second constrained variable volume 128 is positionable to overlap all of the plurality of second inlet ports 112 of the second collapsible fluid reservoir 68 being independent of an overlap with an inlet port in fluid communication with another collapsible fluid reservoir.

FIGS. 14A-14D illustrate delivery sequence and venting sequence embodiments. A delivery sequence embodiment may be initiated in some cases by first filling the second constrained variable volume 128 from the second collapsible fluid reservoir 68 through second inlet port 112. With the second constrained variable volume 128 in fluid communication with the second inlet port 112, the drive mechanism 48 may be actuated to apply axial tension on the first spool section 136, which is then transmitted to the second spool section 138, as shown by the arrow 161 in FIG. 14A. FIG. 14B shows the second constrained variable volume 128 completely filled with fluid from the second collapsible fluid reservoir 68. During the fill step, the third spool section 146 may remain substantially stationary and the second limited displacement coupling 158 is axially expanded, as to increase the second constrained variable volume 128. Fluid is drawn into the second volume, as indicated by arrow 163 in FIG. 14B. As shown, an enlarged portion 166 of the axial extension 162 of the second limited displacement coupling 158 may have been displaced to a position adjacent a shoulder portion of the cavity 168 of the second limited displacement coupling 158. It should be noted that the first limited displacement coupling 156 may have remained in an axially contracted configuration during the fill process of the second constrained variable volume 128, because the first constrained variable volume 122, between the first spool section 136 and second spool section 138, is not in fluid communication with any inlet or outlet port; thus, the volume may remain hydraulically locked together.

Once the second constrained variable volume 128 has been filled, the third spool section 146 is proximally retracted further, or displaced until the first constrained variable volume 122 comes into fluid communication with the first inlet port 108, as shown in FIG. 14C. Once the first constrained variable volume 122 is in fluid communication with the first inlet port 108, the negative pressure caused by the axial friction and drag of the seals of the second spool section 138 and first spool section 136 causes the first constrained variable volume 122 to begin filling, as shown by the arrow 165 in FIG. 14C. The first constrained variable volume 122 continues to fill with the fluid from the first collapsible fluid reservoir 70, through the first inlet port 108, until the enlarged portion 160 of the axial extension 164 of the first limited displacement coupling 156 is disposed, adjacent to the shoulder portion of the cavity 159 of the first limited displacement coupling 156, as shown in FIG. 14D. At this stage, the three spool sections 136, 138, and 146 are mechanically coupled with both of the limited displacement couplings 156 and 158 fully extended, as shown in FIG. 14D.

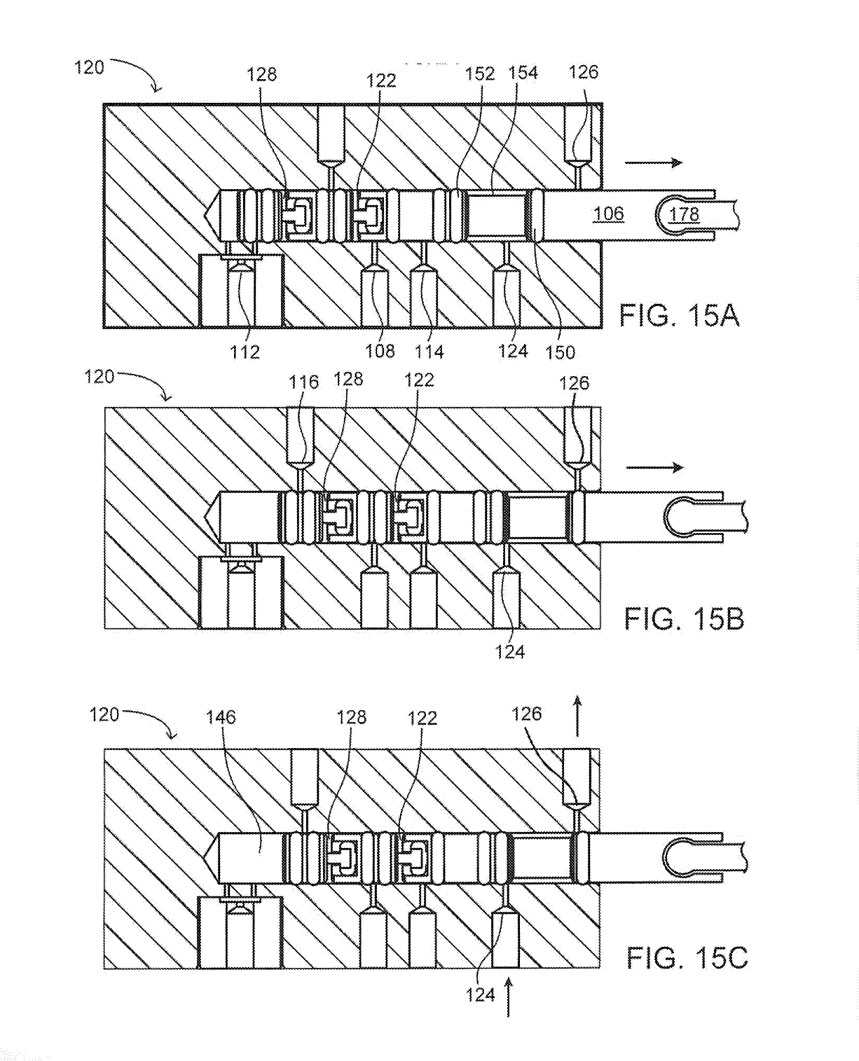

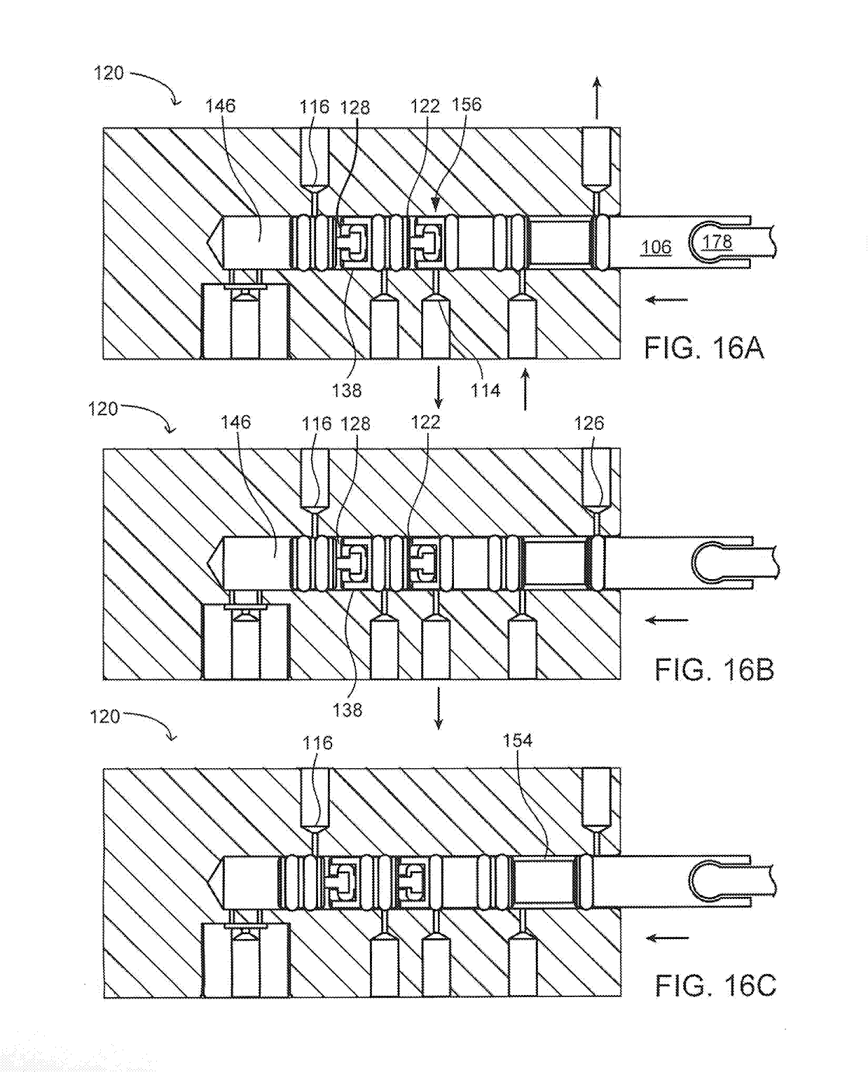

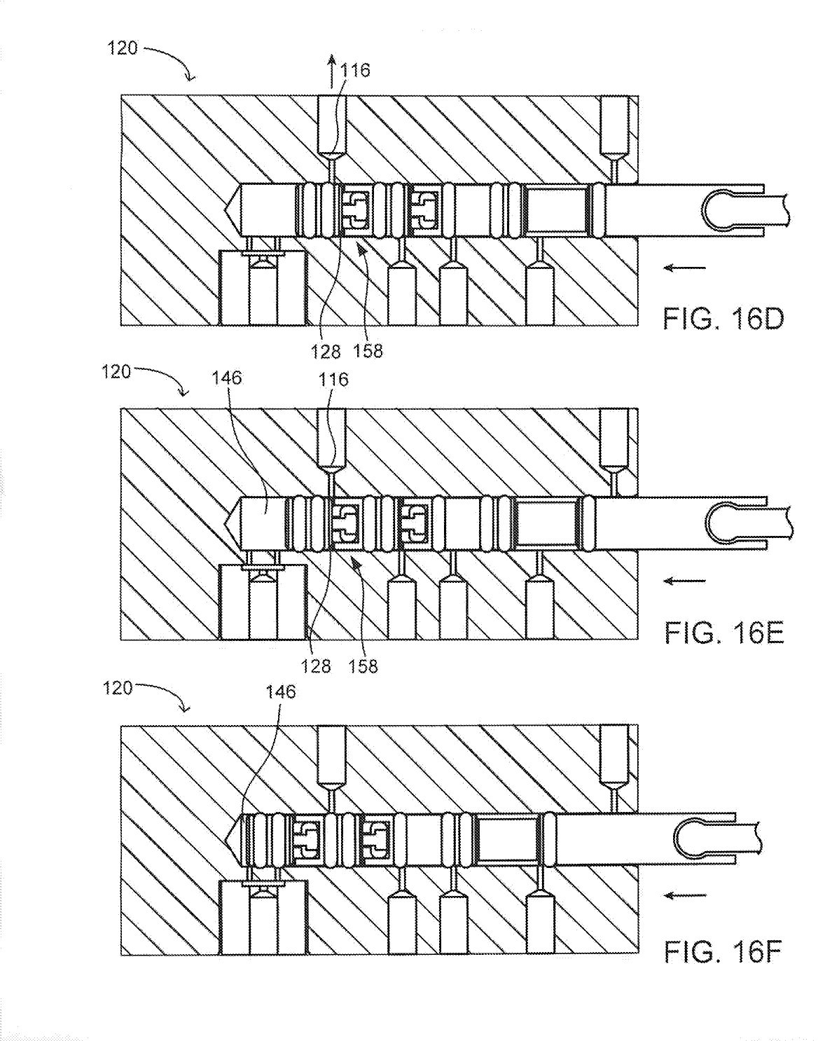

With both of the constrained variable volumes 122 and 128 completely filled, a venting cycle is initiated by further displacing the spool 106 axially, in a proximal direction, as shown in FIG. 15A. Proximal retraction or displacement of the spool 106 continues, as shown in FIG. 15B, with the second vented volume 154 of the spool 106 formed between a first vent seal 150 and a second vent seal 152, is disposed adjacent to the vent inlet port 124 and vent outlet port 126. As the spool 106 is further translated in a proximal direction, the second vented volume 154, formed between the first vent seal 150 and second vent seal 152, becomes disposed in fluid communication with both the vent inlet port 124 and vent outlet port 126; so as, to create a vent conduit extending from the vent inlet port 124 to the ambient atmosphere outside the vent outlet port 126, as shown in FIG. 15C. Air or any other fluid disposed within the first vented volume 80 may be vented to the atmosphere, as shown by the arrows in FIG. 16C. Air or any other fluid from the ambient atmosphere (or any other volume in fluid communication with the vent outlet port 126) may also be vented inward into the first vented volume 80, in a direction opposite to that of the vent arrows shown in FIG. 15C. Such a venting process may be typical, as fluid is withdrawn from the first and the second collapsible fluid reservoirs 70 and 68, air from the ambient atmosphere may be drawn into the first vented volume 80, in order to fill the void left in the first vented volume 80 by the dispensed fluid.

Once the first vented volume 80 has been vented, or at any other suitable time prior to venting, the drive mechanism 48 may begin to apply a distal axial force to the spool 106; and in particular, to the proximal end of the first spool section 136, as shown in FIG. 16A. As the axial force from the drive mechanism 48 is exerted onto the first spool section 136, each of the first and second constrained variable volumes 128 and 122 are compressed, due to the friction of the seals of the second spool section 138 and third spool section 146, which increases the pressure of the fluid in the filled volumes. Because the first constrained variable volume 122 is in fluid communication with the first outlet port 114 at this stage, the increased pressure within the first constrained variable volume 122 delivers the fluid from the first constrained variable volume 122 to the first outlet port 114, as shown by the arrows in FIGS. 16A and 16B. The delivery process of fluid from the first constrained variable volume 122 continues until the first limited displacement coupling 156 bottoms out, and the first spool section 136 comes into solid mechanical contact with the second spool section 138, as shown in FIG. 16B. As distal axial force is further applied, the spool 106 begins to advance together in a distal direction, until the second constrained variable volume 128 comes into fluid communication with the second outlet port 116, as shown in FIG. 16C. Once the second constrained variable volume 128 is in fluid communication with the second outlet port 116, fluid may begin to be dispensed or delivered from the second constrained variable volume 128. The delivery of fluid from the second constrained variable volume 128 may continue through the second outlet port 116 until the second limited displacement coupling 158 bottoms out, and the third spool section 146 begins to advance in a distal direction, as shown in 16E. The distal advancement of the spool 106 may continue until the spool 106 is returned to the home position, as shown in FIG. 16F, at which point, the delivery and vent cycle may begin again.

It should be noted that while fluid was delivered from both the first collapsible fluid reservoir 70 and second collapsible fluid reservoir 68 for the delivery cycles discussed above, fluid may also be delivered from each of the collapsible fluid reservoirs 70 and 68 respectively, and independently of delivery of fluid from the other collapsible fluid reservoir. For example, the second constrained variable volume 128 may be repeatedly shuttled back and forth from a position in fluid communication with the second inlet port 112, to a position in fluid communication with the second outlet port 116, without ever making a fluid delivery to the first outlet port 114. The second constrained variable volume 128 may not overlap the first section 132 of the axial bore 104 and vice versa. As such, there may be no cross-contamination between the fluid within the first collapsible fluid reservoir 70 and the fluid within the second collapsible fluid reservoir 68. As discussed above, in some cases the first collapsible fluid reservoir 70 may contain a first therapeutic fluid for delivery to the patient 300, and the second collapsible fluid reservoir 68 may contain a second therapeutic fluid for delivery to the patient. For instance, in some cases, the first therapeutic fluid may include a fast acting insulin compound and the second therapeutic fluid may include a slow acting insulin compound.

In other embodiments, an infusion system may include an infusion pump and an infusion set. In some cases the infusion pump may include a delivery mechanism having a first delivery section which includes a first inlet port, a first outlet port and a first constrained variable volume that is translatable between a position in fluid communication with the first inlet port and a position in fluid communication with the first outlet port. The delivery mechanism may also include a second delivery section which includes a second inlet port, a second outlet port, and a second constrained variable volume that is translatable between a position in fluid communication with the second inlet port and a position in fluid communication with the second outlet port.

The infusion pump of such embodiments may further include a first fluid reservoir including an interior volume in fluid communication with the first inlet port and a second fluid reservoir including an interior volume in fluid communication with the second inlet port and a drive mechanism. The drive mechanism may be operatively coupled to the first constrained variable volume and operatively coupled to the second constrained variable volume. In some such cases, the drive mechanism may be configured to translate the first constrained variable volume between the first inlet port and first outlet port and configured to expand the first constrained variable volume while in fluid communication with the first inlet port and contract the first constrained variable volume while in fluid communication with the first outlet port. The drive mechanism may also be configured to translate the second constrained variable volume between the second inlet port and second outlet port and configured to expand the second constrained variable volume while in fluid communication with the second inlet port and contract the second constrained variable volume while in fluid communication with the second outlet port. In some cases, the drive mechanism may also be configured to expand or contract the first constrained variable volume by exerting translational force through a boundary section of the first constrained variable volume and configured to expand or contract the second constrained variable volume by exerting translational force through a boundary section of the second constrained variable volume.

In some embodiments, the infusion set may be configured to be attached to a patient's skin and to be coupled to the infusion pump. The infusion set may also include a dual-lumen cannula that is adapted to deliver medications in subcutaneous spaces under the skin of the patient. The dual-lumen cannula may include a first cannula and a second cannula. The first cannula may be configured to provide a first therapeutic fluid and the second cannula may be configured to provide a second therapeutic fluid. The first cannula and the second cannula of the dual-lumen cannula may be joined together in some cases to form a dual cannula assembly with a first and a second lumen. The first and the second cannulas may have separate first and second proximal ends respectively. A first cannula inlet port and a second cannula inlet port may be formed at the first and the second proximal ends respectively. For some embodiments, a length of the first cannula exceeds a length of the second cannula such that the distal ends of the first and the second cannulas are offset from each other. The first cannula inlet port is coupled to the first outlet port of the infusion pump, and the second cannula inlet port is coupled to the second outlet port of the infusion pump.

Other embodiments include an infusion set configured to be attached to a patient's skin and to be coupled to an infusion pump. The infusion set may include a dual-lumen cannula adapted to deliver medications in subcutaneous spaces under the skin of the patient. The dual-lumen cannula may include a cannula housing and a connector assembly. The cannula housing may include a top shell having two side-access ports. Two self-sealing septa may be disposed in a bottom portion of the cannula housing. The self-sealing septa may each comprise an elastomeric material in some cases. A catheter may be disposed under each of the self-sealing septa. The catheters may be configured to penetrate the skin of a patient. The connector assembly is removably connected to the cannula. The connector assembly includes a first and a second connector assembly inlet port. A first connecting needle may be embedded into the first connector assembly inlet port and a second connecting needle may be embedded into the second connector assembly inlet port. Distal ends of the connecting needles are configured to mate with and to pierce into the self-sealing septa to form fluid passageways to the catheters. In some cases, fluid tubing is adapted to connect the inlet ports of the connector assembly to the outlet ports of a fluid delivery device.

Some embodiments may be directed to an infusion set configured to be attached to a patient's skin and to be coupled to the infusion pump. The infusion set may include a dual-lumen cannula adapted to deliver medications in subcutaneous spaces under the skin of the patient. The dual-lumen cannula may include a cannula housing and a connector assembly. The cannula housing may include a top shell having at least one top-access port and one side-access port. A first and a second self-sealing septum may be disposed in a bottom portion of the cannula housing. In some cases, the top-access port may be configured to allow a single needle to pierce into the first self-sealing septum. A catheter is disposed under each of the self-sealing septa. The single needle through the first self-sealing septum forms a fluid passageway to one of the catheters which may be configured to penetrate the skin of a patient. A connector assembly is adapted to be removably connected to the cannula housing. The connector assembly includes a connecting needle which is embedded into the connector assembly inlet port. A distal end of the connecting needle may be configured to mate with and to pierce into the self-sealing septum to form a fluid passageway to the other catheter. The connecting needle and the single needle are connected to fluid tubing to provide fluid passageways to the catheters.

Some embodiments include a method of delivering two different fluids to a patient from a single device having two independent fluid reservoirs. In some cases, such methods may include providing an infusion pump that has a delivery mechanism with a first inlet port, a second inlet port spaced from the first inlet port, and at least one outlet port which is spaced from the first inlet port and second inlet port. The delivery mechanism may also have a constrained variable volume translatable between a position in fluid communication with the first inlet port, a position in fluid communication with the second inlet port and a position in fluid communication with the outlet port. The infusion pump may further include a first fluid reservoir including an interior volume in fluid communication with the first inlet port, a second fluid reservoir including an interior volume in fluid communication with the second inlet port and a drive mechanism which is operatively coupled to the constrained variable volume that is configured to axially translate the constrained variable volume from each of the inlet ports to one or more outlet ports and which is configured to expand or contract the constrained variable volume of the spool. A dispense cycle may be initiated by translating the constrained variable volume into a position in fluid communication with the first inlet port. A translational force may then be exerted through a boundary section of the constrained variable volume to expand the constrained variable volume and draw a first fluid into the constrained variable through the first inlet port from the first reservoir. The constrained variable volume may then be translated into a position in fluid communication with a first outlet port. Translational force exerted through a boundary section of the constrained variable volume at least partially contracts the constrained variable volume to dispenses the first fluid from the constrained variable volume through the first outlet port and through a first lumen of a dual-lumen cannula to the patient. In addition, the constrained variable volume may be translated to a position in fluid communication with the second inlet port and a translational force exerted through a boundary section of the constrained variable volume can expand the constrained variable volume and draw a second fluid into the constrained variable through the second inlet port from the second reservoir. The constrained variable volume may then be translated to a position in fluid communication with a second outlet port and a translational force exerted through a boundary section of the constrained variable volume at least partially contracts the constrained variable volume and dispenses the second fluid from the constrained variable volume through the second outlet port and through a second lumen of the dual-lumen cannula to the patient. In some such embodiments, the first fluid reservoir may contain a first therapeutic agent for delivery to a patient and the second fluid reservoir may contain a second therapeutic agent different from the first therapeutic agent for delivery to a patient.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet, these modifications and improvements are within the scope and spirit of the technology. The technology illustratively and suitably described herein, may be practiced in the absence of any element(s) not specifically disclosed herein. Thus, for example, in each instance herein, any of the terms “comprising”, “consisting essentially of”, and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” may refer to one of or a plurality of the elements it modifies (e.g., “a reagent” can mean one or more reagents) unless it is contextually clear, either one of the elements, or more than one of the elements is described. Although the present technology has been specifically disclosed by representative embodiments and optional features, modification and variation of the concepts herein disclosed may be made, and such modifications and variations may be considered within the scope of this technology.

What is claimed is:

1. An ambulatory infusion system comprising:
an infusion cartridge including a rigid housing, a collapsible fluid reservoir formed from flexible material and disposed within the rigid housing, a dispense mechanism fluidly connected to the fluid reservoir and an outlet port fluidly connected to the dispense mechanism;
a length of tubing fixedly connected to the outlet port of the infusion cartridge, the length of tubing in fluid communication with the outlet port of the infusion cartridge; and
a connector operably connected to the length of tubing at a terminus of the tubing, the tubing extending as a unitary construct from the outlet port of the infusion cartridge to the connector, the connector adapted to connect to a cannula and/or needle to permit a fluid drug to flow from the infusion cartridge, through the length of tubing, and into the cannula and/or needle.

2. The ambulatory infusion system of claim 1, wherein the length of tubing has a constant, uninterrupted outer diameter between the infusion cartridge and the connector.

3. The ambulatory infusion system of claim 1, wherein the length of tubing includes no connectors between the infusion cartridge and the connector.

4. The ambulatory infusion system of claim 1, wherein the length of tubing cannot be detached from the cartridge without damaging the length of tubing.

5. The ambulatory infusion system of claim 1, wherein the length of tubing is fixedly attached to the cartridge with an adhesive.

6. The ambulatory infusion system of claim 1, further comprising a needle and/or cannula adapted to be attached to the connector.

7. The apparatus of claim 1, wherein the length of tubing comprises more than one lumen.

8. The apparatus of claim 7, wherein the length of tubing comprises coaxial lumens.

9. An infusion kit comprising:
a cannula comprising a first end configured to pierce a patient's skin and a second end;
an infusion cartridge comprising—
a rigid housing;
a collapsible fluid reservoir formed from a flexible material and disposed within the rigid housing;
a dispense mechanism fluidly connected to the fluid reservoir;
an outlet port fluidly connected to the dispense mechanism; and
an infusion set comprising—
a length of tubing fixedly connected to the outlet port of the infusion cartridge, the length of tubing in fluid communication with the outlet port of the infusion cartridge; and
a connector operably connected to the length of tubing at a terminus of the tubing, the tubing extending as a unitary construct from the outlet port of the infusion cartridge to the connector, the connector adapted to connect to the cannula to permit a fluid drug to flow from the infusion cartridge, through the length of tubing, and into the cannula via the second end of the cannula.

10. The kit of claim 9, further comprising an insertion mechanism configured to insert the cannula under the skin of a patient.

11. The kit of claim 9, further comprising a pump.

12. The kit of claim 11, wherein the pump comprises a drive mechanism that functionally and operably interfaces with the dispense mechanism.

13. The kit of claim 12, wherein the drive mechanism regulates flow of medicament from the collapsible fluid reservoir outwards through the outlet port.

14. The kit of claim 9, wherein the length of tubing has a constant, uninterrupted outer diameter between the infusion cartridge and the connector.

15. The kit of claim 9, wherein the length of tubing includes no connectors between the infusion cartridge and the connector.

16. The kit of claim 9, wherein the length of tubing cannot be detached from the cartridge without damaging the length of tubing.

17. The kit of claim 9, wherein the length of tubing is fixedly attached to the cartridge with an adhesive.

18. The kit of claim 9, wherein the length of tubing comprises more than one lumen.

19. The kit of claim 18, wherein the length of tubing comprises coaxial lumens.
